(19) United States
(12) Patent Application Publication
(10) Pub. No.: US 2008/0229521 A1
(43) Pub. Date: Sep. 25, 2008

(54) Process for protecting the color of artificially dyed keratin fibers with respect to washing and light; dyeing processes

(76) Inventor: Boris Lalleman, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/976,642

(22) Filed: Oct. 26, 2007

Related U.S. Application Data
(60) Provisional application No. 60/856,011, filed on Nov. 2, 2006.

(30) Foreign Application Priority Data
Oct. 26, 2006 (FR) 0654562
Oct. 26, 2006 (FR) FR 0654563

(51) Int. Cl. A61K 8/18; A61K 8/27; A61Q 5/10; A61K 8/23; A61Q 5/12

(52) U.S. Cl. 8/408; 8/442; 8/406

(57) ABSTRACT

The invention relates to a process for protecting the colour of artificially dyed keratin fibres with respect to washing and atmospheric agents, characterized in that it consists in applying at least:
a) a composition (B) comprising, in a cosmetically acceptable medium, at least one agent for protection against the effects of atmospheric agents, in particular the light,
b) and a composition (C) comprising, in a cosmetically acceptable medium, at least one zinc-based compound; it being possible for the composition (C) to be applied before or after dyeing of the keratin fibres, and the composition (B) being applied after dyeing of the keratin fibres.

The invention also relates to a process for dyeing keratin fibres, in particular human keratin fibres, and more particularly the hair, consisting in applying to said fibres at least:
(i) a direct dye composition (A) or an oxidation dye composition (A) in the presence of an oxidizing agent for a period of time sufficient to develop the colour,
(ii) a composition (B) as defined above,
(iii) a composition (C) as defined above;
   it being possible for the composition (C) to be applied before or after the keratin fibre dyeing step, and the composition (B) being applied after the keratin fibre dyeing step.

This application claims benefit of U.S. Provisional Application No. 60/856,011, filed Nov. 2, 2006, the contents of which are incorporated herein by reference. This application also claims benefit of priority under 35 U.S.C. § 119 to French Patent Application No. FR 0654563, filed Oct. 26, 2006, the contents of which are also incorporated herein by reference.

The present disclosure relates to a process for protecting the color of artificially dyed keratin fibers with respect to washing and atmospheric agents. This process comprises applying to artificially dyed keratin fibers at least:

a) a composition (B) comprising, in a cosmetically acceptable medium, at least one agent for protection against the effects of atmospheric agents, e.g., light; and

b) a composition (C) comprising, in a cosmetically acceptable medium, at least one zinc-based compound;

wherein composition (C) may be applied before or after dyeing of the artificially dyed keratin fibers, and composition (B) is applied after dyeing of the artificially dyed keratin fibers.

It is known practice to dye hair with dye compositions containing oxidation dye precursors, which are generally known as oxidation bases. These oxidation bases are colorless or weakly colored compounds which, when combined with oxidizing products, give rise to colored compounds via oxidative condensation. It is also known that the shades obtained with these oxidation bases may be varied by combining them with couplers or coloration modifiers. The variety of molecules used as oxidation bases and couplers allows a wide range of colors to be obtained.

It is also known practice to dye hair by direct dyeing. The process conventionally used in direct dyeing comprises applying to the hair direct dyes, which are colored and coloring molecules that have an affinity for the hairleaving them to act, and then rinsing the fibers.

The colorings resulting from direct dyeing are particularly chromatic, but are also, however, temporary or semi-permanent due to the nature of the interactions that bind the direct dyes to the keratin fiber, and their desorption from the surface and/or the core of the fiber. As a result, direct dyes may have weak dyeing power and poor wash-fastness.

It is also known that the artificial color of the hair provided by a direct or oxidation dyeing treatment gradually fades as a result of repeated washing and exposure to the light, leading over time to fading of the coloring of the hair.

Even though considerable progress has been made to improve the protection of artificial hair color, for example by using protective agents in combination with an aromatic alcohol and an aromatic carboxylic acid, as described in European Patent Application EP 1 688 127, there still exists a need to further limit the fading of the artificial color of the hair.

It would therefore be desirable to develop means for improving the fastness of artificial color, for example with respect to the effect of repeated washing and of light, in a long-lasting manner.

Organic mineral zinc salts are generally known in hair dyeing as catalysts for oxidation dyeing with atmospheric oxygen using oxidation dye precursors. In European Patent EP 335 403, for example, zinc salts are used for mild oxidation dyeing of the hair with tannins, such as catechin or tannic acid. Further, in French Patent Application FR 2 814 943 and European Patents EP 1 424 060 and EP 1 210 931, zinc salts are used in a catalytic system that also comprises hydrogen carbonates for developing the color of ortho-diphenol type oxidation dye precursors. German Patent Application DE 42 098 97 describes processes for oxidation dyeing based on indol or indoline compounds and zinc salts.

Japanese Application JP 2003-095897 describes treatment compositions for preventing problems with the shade of discolored hair caused by light, shampoo at the time of dyeing, or the use of peroxide. These compositions contain a hydroxycarboxylic acid chosen, for instance, from copper gluconate, zinc gluconate and gallic acid 2-glucoside.

The present inventor has now discovered, surprisingly, that by applying to keratin fibers, after direct or oxidation dyeing, simultaneously or one after the other, a composition C comprising at least one zinc-based compound and a composition B comprising at least one agent for protecting against the effect of atmospheric agents, the fastness of the color after washing and/or exposure to atmospheric agents such as light, is substantially improved.

Furthermore, the protection provided by the treatment according to the present disclosure is long-lasting, i.e., does not require frequent reapplications of the product.

Therefore, the present disclosure relates, in one non-limiting aspect, to a process for protecting the color of artificially dyed keratin fibers with respect to washing and atmospheric agents, comprising the application of at least:

a) a composition (B) comprising, in a cosmetically acceptable medium, at least one agent for protection against the effects of atmospheric agents, such as light;

b) at least one composition (C) comprising, in a cosmetically acceptable medium, at least one zinc-based compound;

wherein composition (C) may be applied before or after dyeing of the artificially dyed keratin fibers, and composition (B) is applied after dyeing of the keratin fibers.

Another non-limiting aspect of the present disclosure concerns a dyeing process comprising applying to keratin fibers, e.g., human keratin fibers such as the hair:

(i) a direct and/or oxidation dye composition (A) in the presence of an oxidizing agent, for a period of time sufficient to develop the color,

(ii) a composition (B) comprising, in a cosmetically acceptable medium, at least one agent for protection against the effects of atmospheric agents, such as light,

(iii) a composition (C) comprising, in a cosmetically acceptable medium, at least one zinc-based mineral compound and/or a zinc-based non-nitrous organic compound;

wherein composition (C) may be applied before or after application of the direct and/or oxidation dye composition, and composition (B) is applied after application of the direct and/or oxidation dye composition.

As used herein, the term:
“oxidizing agent,” means any compound having oxidizing properties and being different from oxygen from the air;
“zinc-based compound” means any inorganic or organic compound comprising in its structure at least one zinc atom;
“human keratin fibers” means the hair of the head, body hairs, such as beard or moustache hairs, the eyelashes or the eyebrows;
“artificially dyed keratin fibers” means keratin fibers dyed by a direct dyeing process or by an oxidation dyeing process in the presence of an oxidizing agent; and
“washing” means at least one application of an aqueous rinse-out composition, such as a detergent composition, e.g., a shampoo, to keratin fibers. This expression also includes bathing, e.g., in the sea or in a swimming pool.

The various aspects of the present disclosure are described in detail below. All the meanings and definitions of the compounds used in accordance with the present disclosure are valid for all aspects of the present disclosure.

The cosmetically acceptable medium of compositions (B) and (C) for protecting the color according to the present disclosure may, for example, comprise water or a mixture of water and at least one cosmetically acceptable organic solvent. As examples of suitable organic solvents, non-limiting mention may be made of C₁-C₄ lower alkanols, such as ethanol and isopropanol; polyols and polyol ethers, such as 2-butoxyethanol, propylene glycol, propylene glycol monomethyl ether, diethylene glycol monoethyl ether and diethylene glycol monomethyl ether, and mixtures thereof.

The solvents may be present in an amount ranging from 1% to 40% by weight relative to the total weight of the composition (B) or (C), for example, from 3% to 10% by weight.

The at least one agent for protection of the keratin fibers present in composition (B) may be any active agent that can be used for preventing or limiting degradation of keratin fibers, such as hair, due to atmospheric effects, such as from light.

Accordingly, the agent for protection of keratin fibers may, for example, be chosen from organic UV screening agents, free-radical scavengers, and antioxidants.

As used herein, the term, “free-radical scavenger,” means any compound capable of trapping free radicals.

Non-limiting examples of free-radical scavengers that can be used in the composition according to the present disclosure include, in addition to certain anti-pollution agents previously mentioned, vitamin E and its derivatives, such as tocopheryl acetate; bioflavonoids; coenzyme Q10 or ubiquinone; certain enzymes, such as catalase, superoxide dismutase and wheat germ extracts containing the same, lactoperoxidase, glutathione peroxydase and quinone reductases; glutathione; benzylidenecamphor; benzylcyclanones; substituted naphthalenones; pidolates; phytantriol; gamma-oryzanol; guanosine; lignans; and melatonin.

Antioxidants that may be used as the agent for protection of keratin fibers include, for example, phenols, such as BHA (tert-butyl-4-hydroxyanisole), BHT (2,6-di-tert-butyl-p-cresol), TBHQ (tert-butylhydroquinone), polyphenols such as proanthocyanidol oligomers and flavonoids, hindered amines known under the generic term HALS (Hindered Amine Light Stabilizer) such as tetraminopiperidine, erythorbic acid, polyamines such as spermine, cysteine, glutathione, superoxide dismutase or lactoferrin.

The organic UV screening agents (systems for screening out UV radiation) may, for example, be chosen from water-soluble or liposoluble, silicone or non-silicone screening agents.

Non-limiting examples of organic screening agents that may be used include dibenzoylmethane derivatives; anthranilates; cinnamic derivatives; salicylic derivatives; camphor derivatives; benzophenone derivatives; β,β-diphenylacrylate derivatives; triazine derivatives; benzotriazole derivatives; benzalmalonate derivatives; benzimidazole derivatives; imidazolines; bis-benzoazolyl derivatives as described in European Patent EP 669 323 and U.S. Pat. No. 2,463,264; p-aminobenzoic acid (PABA) derivatives; benzoxazole derivatives, e.g., those described in European Patent Applications EP 0 832 642, EP 1 027 883, and EP 1 300 137 and German Patent Application DE 101 62 844; screening polymers and screening silicones, such as those described in International Application WO 93/04665; dimers derived from α-alkylstyrene, such as those described in German Patent Application DE 198 55 649; 4,4-diarylbutadienes, such as those described in German Patent Applications DE 197 46 654 and DE 197 55 649 and European Patent Applications EP 0 967 200, EP-A-1 008 586, EP 1 133 980 and EP 133 981, and mixtures thereof.

As examples of organic UV screening agents, non-limiting mention may be made of the following (denoted by INCI name):

### Para-Aminobenzoic Acid Derivatives:

### PABA,

### Ethyl PABA,

Ethyl dihydroxypropyl PABA,
Ethyhexyl dimethyl PABA sold, e.g., under the name “Escalol 507” by ISP,

### Glyceryl PABA,

PEG-25 PABA sold under the name “Uvinul P25” by BASF.

### Cinnamic Derivatives:

Ethylhexyl methoxycinnamate sold, e.g., under the trade name “Parsol MCX” by Hoffmann LaRoche,
Isopropyl methoxycinnamate,
Isoamyl methoxycinnamate sold under the trade name “Neo Heliopan E 1000” by Haarmann and Reimer,

### Cinoxate,

DEA methoxycinnamate,
Diisopropyl methylcinnamate,
Glyceryl ethylhexanoate dimethoxycinnamate.

### Dibenzoylmethane Derivatives:

Butylmethoxydibenzoylmethane sold, e.g., under the trade name “Parsol 1789” by Hoffmann LaRoche,
Isopropyldibenzoylmethane sold, e.g., under the trade name “Eusolex 8020” by Merck.

### Salicylic Derivatives:

Homosalate sold under the name “Eusolex HMS” by Rona/EM Industries,
Ethylhexyl salicylate sold under the name “Neo Heliopan OS” by Haarmann and Reimer,
Dipropylene glycol salicylate sold under the name “Dipsal” by Scher,
TEA salicylate sold under the name “Neo Heliopan TS” by Haarmann and Reimer.

### β,β-Diphenylacrylate Derivatives:

Octocrylene sold in particular under the trade name “Uvinul N539” by BASF,
Etocrylene sold in particular under the trade name “Uvinul N35” by BASF.

### Benzophenone Derivatives:

Benzophenone-1 sold under the trade name “Uvinul 400” by BASF,
Benzophenone-2 sold under the trade name “Uvinul D50” by BASF,
Benzophenone-3 or Oxybenzone sold under the trade name “Uvinul M40” by BASF,
Benzophenone-4 sold under the trade name “Uvinul MS40” by BASF,

### Benzophenone-5,

Benzophenone-6 sold under the trade name “Helisorb 11” by Norquay,
Benzophenone-8 sold under the trade name “Spectra-Sorb UV-24” by American Cyanamid,
Benzophenone-9 sold under the trade name “Uvinul DS-49” by BASF,

### Benzophenone-12

n-hexyl 2-(4-diethylamino-2-hydroxybenzoyl)benzoate.

### Benzylidenecamphor Derivatives:

3-Benzylidenecamphor manufactured under the name “Mexoryl SD” by Chimex,
4-Methylbenzylidenecamphor sold under the name “Eusolex 6300” by Merck,
Benzylidenecamphorsulphonic acid manufactured under the name “Mexoryl SL” by Chimex,
Camphor benzalkonium methosulphate manufactured under the name “Mexoryl SO” by Chimex,
Terephthalylidenedicamphorsulphonic acid manufactured under the name “Mexoryl SX” by Chimex,
Polyacrylamidomethylbenzylidenecamphor manufactured under the name “Mexoryl SW” by Chimex.

### Phenylbenzimidazole Derivatives:

Phenylbenzimidazolesulphonic acid sold, e.g., under the trade name “Eusolex 232” by Merck,
Disodium phenyl dibenzimidazole tetrasulphonate sold under the trade name “Neo Heliopan AP” by Haarmann and Reimer.

### Phenylbenzotriazole Derivatives:

Drometrizole trisiloxane sold under the name “Silatrizole” by Rhodia Chimie,
Methylenebis(benzotriazolyl)tetramethylbutylphenol sold in solid form under the trade name “MIXXIM BB/100” by Fairmount Chemical, or in micronized form as an aqueous dispersion under the trade name “Tinosorb M” by Ciba Specialty Chemicals.

### Triazine Derivatives:

Bisethylhexyloxyphenol Methoxyphenyl Triazine sold under the trade name “Tinosorb S” by Ciba Geigy, Ethylhexyltriazone sold, e.g., under the trade name “Uvinul T150” by BASF, Diethylhexylbutamidotriazone sold under the trade name “Uvasorb HEB” by Sigma 3V, 2,4,6-tris(diisobutyl 4′-aminobenzalmalonate)-s-triazine.

### Anthranilic Derivatives:

Menthyl anthranilate sold under the trade name “Neo Heliopan MA” by Haarmann and Reimer.

### Imidazoline Derivatives:

Ethylhexyldimethoxybenzylidenedioxoimidazoline propionate.

### Benzalmalonate Derivatives:

Polyorganosiloxane containing benzalmalonate functions, for instance Polysilicone-15, sold under the trade name “Parsol SLX” by Hoffmann LaRoche

### 4,4-Diarylbutadiene Derivatives:

1,1-Dicarboxy(2,2′-dimethylpropyl)-4,4-diphenylbutadiene

### Benzoxazole Derivatives:

2,4-bis[5-(1-dimethylpropyl)benzoxazol-2-yl(4-phenyl)imino]-6-(2-ethylhexyl)imino-1,3,5-triazine sold under the name Uvasorb K2A by Sigma 3V
and mixtures thereof.

As liposoluble (or lipophilic) organic UV screening agents that are suitable for use in the present disclosure, non-limiting mention may be made of:

ethylhexyl methoxycinnamate,
butylmethoxydibenzoyl methane,
homosalate,
ethylhexyl salicylate,
octocrylene,
benzophenone-3,
n-hexyl 2-(4-diethylamino-2-hydroxybenzoyl)benzoate,
4-methylbenzylidenecamphor,
ethylhexyl triazone,
bisethylhexyloxyphenol methoxyphenyl triazine,
diethylhexyl butamido triazone,
drometrizole trisiloxane,
polysilicone-15,
1,1-dicarboxy(2,2′-dimethylpropyl)-4,4-diphenylbutadiene, and
2,4-bis[5-1 (dimethylpropyl)benzoxazol-2-yl(4-phenyl)imino]-6-(2-ethylhexyl)imino-1,3,5-triazine.

As water-soluble (or hydrophilic) organic UV screening agents that are suitable for use in the present disclosure, non-limiting mention may be made of:

### PABA,

### PEG-25 PABA

benzylidenecamphorsulphonic acid,
camphorbenzalkonium methosulphate,
terephthalylidenedicamphorsulphonic acid,
phenylbenzimidazolesulphonic acid,
disodium phenyldibenzimidazoletetrasulphonate,
benzophenone-4, and
benzophenone-5.

According to a non-limiting aspect of the present disclosure, protective agents having a logP (octanol/water partition coefficient) of less than 6, such as less than 4.5, for example less than 2, are used.

According to a further non-limiting aspect of the present disclosure, protective agents which are soluble in the aqueous medium of the composition, such as protective agents soluble at 25° C. at least 0.5% in water or C₁-C₄ lower alcohols such as ethanol, are used.

According to another non-limiting aspect of the present disclosure, protective agents having in their chemical structure at least one acid function in free form or partially or completely salified form, such as a carboxylic acid or sulphonic acid function, are used. For example, protective agents having in their chemical structure at least one sulphonic acid structure in free form or partially or completely salified form may be used.

In addition, in at least one embodiment, a water-soluble organic UV screening agent as defined may be used as the at least one protective agent. In a further non-limiting embodiment, benzophenone-4 is used as a water-soluble organic UV screening agent.

According to the present disclosure, the at least one agent for protection of keratin fibers may be present in an amount ranging from 0.05% to 50% by weight, such from 0.35% to 30% by weight, for example, from 0.5% to 20% by weight, relative to the total weight of composition (B).

According to a non-limiting aspect of the present disclosure, composition (B) further comprises at least one aromatic alcohol and at least one aromatic carboxylic acid.

As used herein, the term, “aromatic alcohol,” means any compound which is liquid at ambient temperature and ambient pressure and which comprises at least one benzene or naphthalene ring and at least one alcohol function (OH) directly attached to the ring or attached to at least one substituent of the ring. In a non-limiting embodiment, the alcohol function is present on a substituent of the benzene or naphthalene ring.

Among the aromatic alcohols that may be used in the composition according to the present disclosure, non-limiting mention may be made of

benzyl alcohol

benzoyl isopropanol

benzyl glycol

phenoxyethanol

dichlorobenzyl alcohol

methylphenylbutanol

phenoxyisopropanol

phenylisohexanol

phenylpropanol

phenylethyl alcohol

mixtures thereof.

In a further non-limiting embodiment, benzyl alcohol is used as the aromatic alcohol.

According to the present disclosure, the at least one aromatic alcohol may be present in an amount ranging from 0.01% to 50% by weight, for example, from 0.1% to 30% by weight, such as from 1% to 20% by weight, relative to the total weight of composition (B). According to at least one non-limiting embodiment, the at least one aromatic alcohol is present in an amount greater than 1% by weight.

The composition (B) according to the present disclosure may further comprise at least one optionally salified aromatic carboxylic acid.

As used herein, the term, “aromatic carboxylic acid,” means any compound comprising at least one benzene or naphthalene ring and at least one carboxylic acid function (COOH), in free or salified form, directly attached to the ring or attached to at least one substituent of said ring. In at least one embodiment, the acid function is directly attached to the benzene or naphthalene ring.

The salts of the aromatic carboxylic acids may be chosen, for example, from alkali metal (sodium, potassium) salts, alkaline earth metal (calcium, magnesium) salts, organic amine salts, or ammonium salts.

Among the aromatic carboxylic acids that can be used in the composition according to the present disclosure, non-limiting mention may be made of

benzoic acid

para-anisic acid

diphenolic acid

ferulic acid

hippuric acid

3-hydroxybenzoic acid

4-hydroxybenzoic acid

phenylthioglycolic acid

acetylsalicylic acid

para-, meta- or ortho-phthalic acid, and also salified forms thereof and mixtures thereof.

In a further embodiment, benzoic acid is used as the aromatic carboxylic acid.

The aromatic acid(s) or salts thereof may be present in an amount ranging from 0.001% to 30% by weight, for example from 0.01% to 20% by weight, such as from 0.1% to 10% by weight, relative to the total weight of composition (B).

The zinc-based compounds may be chosen, for example, from water-soluble zinc salts.

As used herein, the term, “water-soluble zinc salt,” means any salt which, after having been completely dissolved with stirring at 1% in a solution of water at a temperature of 25° C., produces a solution comprising an amount of insoluble salt of less than 0.05% by weight.

Among the water-soluble zinc salts that can be used according to the present disclosure, non-limiting mention may be made of zinc sulphate, zinc chloride, zinc lactate, zinc gluconate, zinc phenolsulphonate, zinc salicylate and its derivatives, or mixtures thereof.

The zinc salicylate and its derivatives according to the present disclosure may, for example, correspond to the following structure:

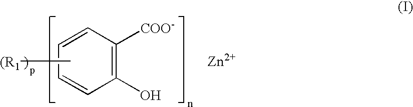

wherein n=2, p is 0, 1, 2 or 3; R¹ is chosen from linear or branched C₁-C₁₈ alkyls (e.g., methyl, ethyl, n-propyl, isopropyl or n-butyl); linear or branched C₁-C₁₈ hydroxyalkyl radicals; halogen atoms (e.g., iodine, bromine or chlorine); C₂-C₁₈ acyl radicals (e.g., acetyl); and COR₂, OCOR₂ or CONHR₂ radicals where R₂ is chosen from a hydrogen atom and a linear or branched C₁-C₁₈ alkyl radical.

According to a non-limiting embodiment of the present disclosure, zinc gluconate, such as the commercial product sold under the name Givobio G Zn by the company Seppic, is used.

The zinc-based compounds in accordance with the present disclosure may be present in the composition (C) in an amount ranging from 0.005% to 30% by weight, for example, from 0.1% to 20% by weight, such as from 0.5% to 15% by weight, relative to the total weight of the composition (C).

Composition (B) and/or composition (C) may further contain various adjuvants conventionally used in hair treatment compositions, such as anionic, cationic, nonionic, amphoteric or zwitterionic surfactants or mixtures thereof, anionic, cationic, nonionic, amphoteric or zwitterionic polymers or mixtures thereof, mineral or organic thickeners, including anionic, cationic, nonionic and amphoteric polymeric associative thickeners, penetrants, sequestrants, fragrances, buffers, dispersants, conditioning agents, for instance modified or unmodified, volatile or non-volatile silicones, film-forming agents, ceramides, preserving agents and opacifiers.

Composition (B) and/or composition (C) may also comprise at least one conditioning agent.

As used herein, the term, “conditioning agent,” means any agent that improves the cosmetic properties of the hair, such as softness, disentangling, feel, smoothness, and static electricity.

The conditioning agents may, for example, be in liquid, semi-solid or solid form, such as, for example, in the form of an oil, wax, or gum.

The conditioning agents may, for example, be chosen from synthetic oils such as polyolefins, plant oils, fluoro or perfluoro oils, natural or synthetic waxes, silicones, non-polysaccharide cationic polymers, compounds of ceramide type, cationic surfactants, fatty amines, saturated fatty acids or fatty acid esters other than those of the present disclosure, and also mixtures of these various compounds.

Non-limiting examples of such synthetic oils include polyolefins, such as poly-α-olefins, including hydrogenated or non-hydrogenated polybutene type polyolefins, such as hydrogenated or non-hydrogenated polyisobutene.

According to at least one non-limiting embodiment, isobutylene oligomers having a molecular weight less than 1000 and mixtures thereof are used together with polyisobutylenes of molecular weight greater than 1000, for example, from 1000 to 15 000.

As examples of poly-α-olefins that may be used according to the present disclosure, non-limiting mention may be made of the polyisobutenes sold under the name PERMETHYL 99 A, 101 A, 102 A, 104 A (n=16) and 106 A (n=38) by the company Presperse Inc, or else the products sold under the name ARLAMOL HD (n=3) by the company ICI (n denoting the degree of polymerization), and of polydecene of the hydrogenated or non-hydrogenated type.

Such products are sold, for example, under the names ETHYLFLO by the company Ethyl Corp., and ARLAMOL PAO by the company ICI.

The animal or plant oils may, for example, be chosen from sunflower oil, maize oil, soya oil, avocado oil, jojoba oil, marrow oil, grapeseed oil, sesame oil, hazelnut oil, fish oils, glyceryl tricaprocaprylate, plant or animal oils of formula R₉COOR₁₀, wherein R₉ is chosen from residues of saturated higher fatty acids containing from 7 to 29 carbon atoms, and R₁₀ is chosen from linear or branched hydrocarbon-based chains containing from 3 to 30 carbon atoms, including alkyl or alkenyl chains, e.g., purcellin oil.

Natural or synthetic essential oils may also be used, including, for example, eucalyptus oil, lavandine oil, lavender oil, vetiver oil, Litsea cubeba oil, lemon oil, sandalwood oil, rosemary oil, camomile oil, savory oil, nutmeg oil, cinnamon oil, hyssop oil, caraway oil, orange oil, geraniol oil, cade oil and bergamot oil.

The waxes may be natural (animal or plant) or synthetic substances that are solid at ambient temperature (20″-25° C.). They are insoluble in water, soluble in oils and are capable of forming a water-repellant film.

A definition of waxes may be found, for example, in P. D. Dorgan, Drug and Cosmetic Industry, December 1983, pp. 30-33.

Non-limiting examples of waxes that may be used according to the present disclosure include, for example, carnauba wax, candelilla wax, alfa wax, paraffin wax, ozokerite, plant waxes such as olive tree wax, rice wax, hydrogenated jojoba wax or absolute waxes of flowers such as the essential oil of blackcurrent flower sold by the company Bertin (France), animal waxes such as beeswaxes, or modified beeswaxes (cerabellina); other waxes or waxy starting materials that can be used according to the present disclosure are, for example, marine waxes such as that sold by the company Sophim under the reference M82, and polyethylene waxes or polyolefin waxes in general.

Conditioning agents that may be used in accordance with the present disclosure include, for example cationic polymers and silicones.

The cationic polymers may be chosen, for example, from cationic polymers that are known per se as improving the cosmetic properties of hair treated with detergent compositions, including, for example, those described in European Patent Application EP-A-0 337 354 and in French Patent Applications FR-A-2 270 846, 2 383 660, 2 598 611, 2470596 and 2519863.

As used herein, the term, “cationic polymer,” means any polymer containing cationic groups and/or groups that may be ionized into cationic groups.

The cationic polymers may be chosen, for example from those which contain units comprising primary, secondary, tertiary and/or quaternary amines that may either form part of the main polymer chain or may be borne by a side substituent directly attached thereto.

The cationic polymers may, for example, have a number-average molecular mass ranging from 500 to 5×10⁶, such as from 10³ to 3×10⁶.

Among the cationic polymers that may be used according to the present disclosure, non-limiting mention may be made of known products such as polymers of the polyamine, polyamino amide and polyquaternary ammonium type, and cationic polymers derived from polysaccharides.

The polymers of the polyamine, polyamido amide and polyquaternary ammonium type that can be used in accordance with the present disclosure include, for example, those described in French Patent Nos. 2 505 348 and 2 542 997. Among these polymers, non-limiting mention may be made of:

Family (1) homopolymers or copolymers derived from acrylic or methacrylic esters or amides and comprising at least one of the units of the following formulae:

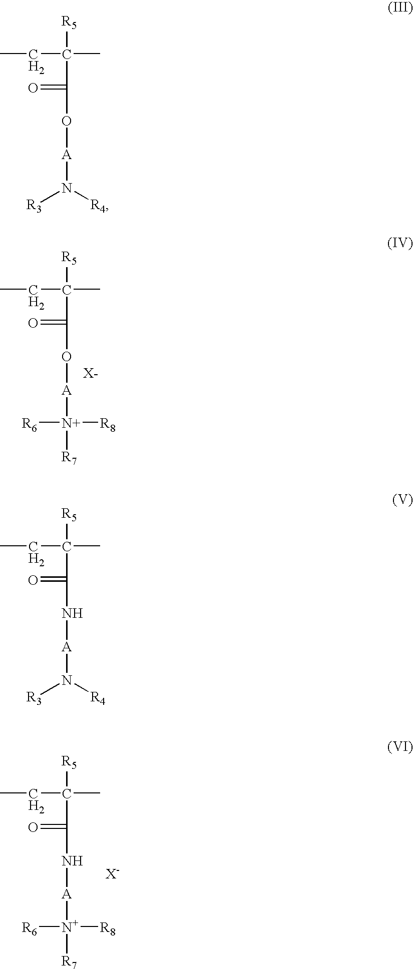

wherein:

R₃ and R₄, which may be identical or different, are independently chosen from hydrogen or alkyl groups containing from 1 to 6 carbon atoms, such as methyl or ethyl;

R₅, which may be identical or different, is chosen from a hydrogen atom or a CH₃ radical;

A, which may be identical or different, is chosen from linear or branched alkyl groups containing 1 to 6 carbon atoms, such as 2 or 3 carbon atoms, and hydroxyalkyl groups containing 1 to 4 carbon atoms;

R₆, R₇ and R₈, which may be identical or different, are each independently chosen from alkyl groups containing from 1 to 18 carbon atoms, e.g., 1 to 6 carbon atoms, and benzyl radicals;

X is chosen from anions derived from a mineral or organic acid, such as a methosulphate anion, or a halide such as chloride or bromide.

The copolymers of family (1) may further comprise at least one unit derived from comonomers that may be chosen from the family of acrylamides, methacrylamides, diacetone acrylamides, acrylamides and methacrylamides substituted on the nitrogen with lower (C₁-C₄) alkyls, acrylic or methacrylic acids or esters thereof, vinyllactams such as vinylpyrrolidone or vinylcaprolactam, and vinyl esters.

Among these copolymers of family (1), non-limiting mention may be made of:

copolymers of acrylamide and of dimethylaminoethyl methacrylate quaternized with dimethyl sulphate or with a dimethyl halide, such as the product sold under the name HERCOFLOC by the company Hercules,

the copolymers of acrylamide and of methacryloyloxyethyltrimethylammonium chloride described, for example, in patent application EP-A-080 976 and sold under the name BINA QUAT P 100 by the company Ciba Geigy,

the copolymer of acrylamide and of methacryloyloxyethyltrimethylammonium methosulphate sold under the name RETEN by the company Hercules,

quaternized or non-quaternized vinylpyrrolidone/dialkylaminoalkyl acrylate or methacrylate copolymers, such as the products sold under the name GAFQUAT by the company ISP, including, for example, GAFQUAT 734 or GAFQUAT 755, or alternatively the products known as Copolymer 845, 958 and 937. These polymers are described in detail in French Patent Nos. 2 077 143 and 2 393 573,

dimethylaminoethyl methacrylate/vinylcaprolactam/vinylpyrrolidone terpolymers, such as the product sold under the name GAFFIX VC 713 by the company ISP,

vinylpyrrolidone/methacrylamidopropyldimethylamine copolymers, such as the product sold under the name STYLEZE CC 10 by ISP, and

quaternized vinylpyrrolidone/dimethylaminopropylmethacrylamide copolymers, such as the product sold under the name GAFQUAT HS100 by the company ISP.

Family (2) Polymers comprising piperazinyl units and divalent alkylene or hydroxyalkylene radicals containing straight or branched chains, optionally interrupted by oxygen, sulphur or nitrogen atoms or by aromatic or heterocyclic rings, as well as the oxidation and/or quaternization products of these polymers. Non-limiting examples of such polymers are described, in French Patent Nos. 2 162 025 and 2 280 361.

Family (3) Water-soluble polyamino amides prepared, for example, by polycondensation of an acidic compound with a polyamine. These polyamino amides may be crosslinked, for example, with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in an amount ranging from 0.025 to 0.35 mol per amine group of the polyamino amide. These polyamino amides may be alkylated or, if they contain at least one tertiary amine function, they may be quaternized. Non-limiting examples of such polymers are described, for example, in French Patent Nos. 2 252 840 and 2 368 508.

Family (4) The polyamino amide derivatives resulting from the condensation of polyalkylene polyamines with polycarboxylic acids followed by alkylation with difunctional agents. Mention may be made, for example, of adipic acid/dialkylaminohydroxy alkyldialkylenetriamine polymers in which the alkyl radical contains from 1 to 4 carbon atoms and, in at least one embodiment, denotes methyl, ethyl or propyl. Such polymers are described, for instance, in French Patent No. 1 583 363.

Among these derivatives, non-limiting mention may be made of the adipic acid/dimethylaminohydroxypropyl/diethylenetriamine polymers sold under the name CARTARETINE F, F4 or F8 by the company Sandoz.

Family (5) The polymers obtained by reaction of a polyalkylene polyamine containing two primary amine groups and at least one secondary amine group with a dicarboxylic acid chosen from diglycolic acid and saturated aliphatic dicarboxylic acids having from 3 to 8 carbon atoms. The molar ratio of polyalkylene polyamine and dicarboxylic acid ranges from 0.8:1 and 1.4:1; the polyamino amide resulting from this reaction is reacted with epichlorohydrin in a molar ratio of epichlorohydrin to the secondary amine group of the polyamino amide ranging from 0.5:1 to 1.8:1. Non-limiting examples of such polymers are described in U.S. Pat. Nos. 3,227,615 and 2 961 347.

Further non-limiting examples of polymers of this type include those sold under the name HERCOSETT 57 by the company Hercules Inc., or alternatively under the name PD 170 or DELSETTE 101 by the company Hercules in the case of the adipic acid/epoxypropyl/diethylenetriamine copolymer.

Family (6) Cyclopolymers of alkyldiallylamine or of dialkyldiallylammonium, such as the homopolymers or copolymers containing, as the main constituent of the chain, units corresponding to formula (VII) or (VIII):

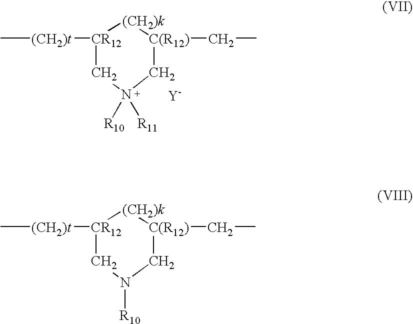

wherein:

k and t equal 0 or 1, with the sum k+t being equal to 1;

R₁₂ is chosen from a hydrogen atom and a methyl radical;

R₁₀ and R₁₁, which may be the same or different, are independently chosen from alkyl groups having from 1 to 6 carbon atoms, hydroxyalkyl groups in which the alkyl group may, for example, have 1 to 5 carbon atoms, and a lower (C₁-C₄) amidoalkyl group, or R₁₀ and R₁₁ may, together with the nitrogen atom to which they are attached, form a heterocyclic group, such as a piperidyl or morpholinyl group;

Y⁻ is chosen from anions such as bromide, chloride, acetate, borate, citrate, tartrate, bisulphate, bisulphite, sulphate or phosphate.

In at least one non-limiting embodiment, R₁₀ and R₁₁, which may be the same or different, are independently chosen from alkyl groups containing 1 to 4 carbon atoms.

Non-limiting examples of polymers of family (6) are described in French Patent No. 2 080 759 and in its Certificate of Addition No. 2 190 406

Among the polymers defined above, non-limiting mention may be of the dimethyldiallylammonium chloride homopolymer sold under the name MERQUAT 100 by the company Nalco (and its homologues of low weight-average molecular mass) and the copolymers of diallyidimethylammonium chloride and of acrylamide, sold under the name MERQUAT 550.

Family (7) The quaternary diammonium polymers containing repeating units, and corresponding to the formula:

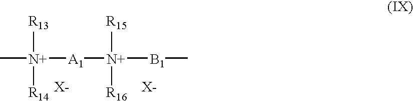

wherein in formula (IX):

R₁₃, R₁₄, R₁₅ and R₁₆, which may be the same or different, are independently chosen from aliphatic, alicyclic or arylaliphatic radicals containing from 1 to 20 carbon atoms, and lower hydroxyalkylaliphatic radicals, or alternatively, R₁₃, R₁₄, R₁₅ and R₁₆, together or separately, constitute, with the nitrogen atoms to which they are attached, heterocycles optionally containing a second heteroatom other than nitrogen, or alternatively R₁₃, R₁₄, R₁₅ and R₁₆ are independently chosen from linear or branched C₁-C₆ alkyl radicals substituted with a nitrile, ester, acyl or amide group or a group —CO—O—R₁₇-D or —CO—NH—R₁₇-D, where R₁₇ is an alkylene and D is a quaternary ammonium group;

A₁ and B₁, which may be the same or different, are independently chosen from polymethylene groups containing from 2 to 20 carbon atoms which may be linear or branched, saturated or unsaturated, and which may contain, linked to or intercalated in the main chain, at least one aromatic rings or at least one oxygen or sulphur atoms or sulphoxide, sulphone, disulphide, amino, alkylamino, hydroxyl, quaternary ammonium, ureido, amide or ester groups;

X⁻ is chosen from anions derived from a mineral or organic acid;

A₁, R₁₃ and R₁₅ can form, together with the two nitrogen atoms to which they are attached, a piperazine ring; further, if A₁ is chosen from a linear or branched, saturated or unsaturated alkylene or hydroxyalkylene radical, B₁ may be shown from a group (CH₂)ₙₚ—CO-D-OC—(CH₂)ₚ—, wherein p is an integer ranging from 2 to 20 and D is chosen from:

a) a glycol residue of formula: —O-Z-O—, where Z is chosen from a linear or branched hydrocarbon-based radical and a group corresponding to one of the following formulae:

—(CH₂—CH₂—O)ₓ—CH₂—CH₂—

—[CH₂—CH(CH₃)—O]_{y}—CH₂—CH(CH₃)—

wherein x and y each represent an integer ranging from 1 to 4, and represent a defined and unique degree of polymerization or any number from 1 to 4 representing an average degree of polymerization;

b) a bis-secondary diamine residue, such as a piperazine derivative;

c) a bis-primary diamine residue of formula: —NH—Y—NH—, where Y is chosen from a linear or branched hydrocarbon-based radical, or alternatively the divalent radical

—CH₂—CH₂—S—S—CH₂—CH₂—;

d) a ureylene group of formula: —NH—CO—NH—.

In at least one non-limiting embodiment, X⁻ is an anion such as chloride or bromide.

Polymers of family (7) may, for example, have a number-average molecular mass ranging from 1000 and 100 000.

Non-limiting examples of polymers of family (7) are described, for example, in French Patent Nos. 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

In at least one non-limiting embodiment of the present disclosure, the polymers of Family (7) correspond to polymers that comprise repeating units corresponding to the formula:

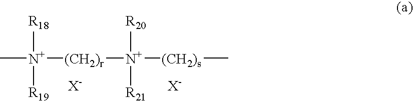

wherein:

R₁₈, R₁₉, R₂₀ and R₂₁, which may be identical or different, are independently chosen from alkyl or hydroxyalkyl radicals containing from 1 to 4 carbon atoms,

r and s are integers ranging from 2 to 20, and

X⁻ is an anion derived from a mineral or organic acid.

A non-limiting example of a compound of formula (a) is the compound for which R₁₈, R₁₉, R₂₀ and R₂₁ each represent a methyl radical, r=3, s=6, and X═Cl, referred to as hexadimethrine chloride according to the INCI nomenclature (CTFA).

Family (8) Polyquaternary ammonium polymers comprising units of formula (X):

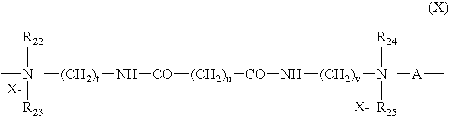

wherein:

R₂₂, R₂₃, R₂₄ and R₂₅, which may be identical or different, are independently chosen from a hydrogen atom or a methyl, ethyl, propyl, β-hydroxyethyl, β-hydroxypropyl or —CH₂CH₂(OCH₂CH₂)ₚOH radical,

p is an integer ranging from 0 to 6, with the proviso that R₂₂, R₂₃, R₂₄ and R₂₅ do not simultaneously represent a hydrogen atom,

t and u, which may be identical or different, are each an integer ranging from 1 to 6,

v is an integer ranging from 0 to 34,

X⁻ is chosen from anions, such as a halides, and

A is chosen from dihalide radicals or, in at least one embodiment, —CH₂—CH₂—O—CH₂—CH₂—.

Non-limiting examples of such compounds are described in European Patent Application EP-A-122 324.

Among these polymers, non-limiting mention may be made, for example, of Mirapol® A 15, Mirapol® AD1, Mirapol® AZ1 and Mirapol® 175 sold by the company Miranol.

Family (9) Quaternary polymers of vinylpyrrolidone and of vinylimidazole, such as, for example, the products sold under the names Luviquat® FC 905, FC 550 and FC 370 by the company BASF.

Family (10) Crosslinked methacryloyloxy(C₁-C₄)alkyltri(C₁-C₄)alkylammonium salt polymers, such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound containing olefinic unsaturation, in particular methylenebisacrylamide. A crosslinked acrylamide/methacryloyloxyethyltrimethylammonium chloride copolymer (20/80 by weight) in the form of a dispersion containing 50% by weight of said copolymer in mineral oil may be used, for example. This dispersion is sold under the name Salcare® SC 92 by the company Ciba. A crosslinked methacryloyloxyethyltrimethylammonium chloride homo-polymer containing about 50% by weight of the homopolymer in mineral oil or in a liquid ester may also be used. These dispersions are sold under the names Salcare® SC 95 and Salcare® SC 96 by the company Ciba.

Family (11) Cationic polysaccharides, such as celluloses and cationic galactomannan gums: Among the cationic polysaccharides, non-limiting mention may be made of cellulose ether derivatives comprising quaternary ammonium groups, cationic polymers of cellulose or cellulose derivatives grafted with a water-soluble quaternary ammonium monomer, and cationic galactomannan gums.

Non-limiting examples of cellulose ether derivatives comprising quaternary ammonium groups are described in French Patent No. 1 492 597. These polymers are also defined in the CTFA dictionary as quaternary ammoniums of hydroxyethylcellulose having reacted with an epoxide substituted with a trimethylammonium group.

Non-limiting examples of cationic copolymers of cellulose or cellulose derivatives grafted with a water-soluble quaternary ammonium monomer are described in U.S. Pat. No. 4,131,576, and include hydroxyalkylcelluloses, such as hydroxymethyl-, hydroxyethyl- or hydroxypropylcelluloses grafted, for example, with a methacryloylethyltrimethylammonium, methacrylamidopropyltrimethylammonium or dimethyldiallylammonium salt.

Non-limiting examples of cationic galactomannan gums are described in U.S. Pat. Nos. 3,589,578 and 4 031 307, and include guar gums containing cationic trialkyl-ammonium groups. Guar gums modified with a 2,3-epoxypropyltrimethylammonium salt (for example, chloride) may be used, for example.

Other non-limiting examples of cationic polymers that can be used include cationic proteins or cationic protein hydrolysates, polyalkyleneimines, such as polyethyleneimines, polymers containing vinylpyridine or vinylpyridinium units, condensates of polyamines and of epichlorohydrin, quaternary polyureylenes, and chitin derivatives.

According to a non-limiting embodiment of the present disclosure, the cationic polymers used are cationic cyclopolymers. As examples of such cationic cyclopolymers, non-limiting mention is made of dimethyldiallylammonium chloride homopolymers and the copolymers sold under the names MERQUAT 100, MERQUAT 550 and MERQUAT S by the company Nalco, quaternary vinylpyrrolidone and vinylimidazole polymers, cationic polysaccharides, and mixtures thereof.

Non-limiting examples of silicones that can be used in accordance with the present disclosure include polyorganosiloxanes that are insoluble in the composition and that may be in the form of oils, waxes, resins or gums.

Organopolysiloxanes are defined, for example, in Walter Noll “Chemistry and Technology of Silicones” (1968) Academic Press. They can be volatile or non-volatile.

If the organopolysiloxanes are volatile, the silicones may be chosen, for example, from those having a boiling point ranging from 60° C. to 260° C. Non-limiting examples of such organopolysiloxanes include:

(i) cyclic silicones containing from 3 to 7, such as from 4 to 5 silicon atoms. These may be, for example, octamethylcyclotetrasiloxane sold in particular under the name VOLATILE SILICONE 7207 by Union Carbide or SILBIONE 70045 V 2 by Rhodia Chimie, decamethylcyclopentasiloxane sold under the name VOLATILE SILICONE 7158 by Union Carbide, and SILBIONE 70045 V 5 by Rhodia Chimie, and mixtures thereof.

Non-limiting mention may also be made of cyclocopolymers of the dimethylsiloxanes/methylalkylsiloxane type, such as VOLATILE SILICONE FZ 3109 sold by the company Union Carbide, and having the chemical structure:

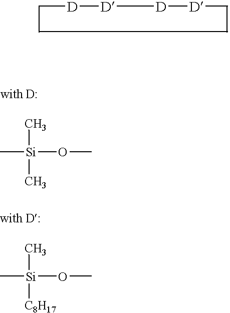

Non-limiting mention may also be made of mixtures of cyclic silicones with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane;

(ii) linear volatile silicones containing 2 to 9 silicon atoms and having a viscosity of less than or equal to 5×10⁻⁶ m²/s at 25° C. A non-limiting example is decamethyltetrasiloxane sold under the name SH 200 by the company Toray Silicone. Silicones belonging to this category are also described in the article published in Cosmetics and Toiletries, Vol. 91, Jan. 76, pp. 27-32, Todd & Byers “Volatile Silicone Fluids for Cosmetics”.

Non-volatile silicones, including polyalkylsiloxanes, polyarylsiloxanes, polyalkylarylsiloxanes, silicone gums and resins, polyorganosiloxanes modified with organofunctional groups, and mixtures thereof, may be used.

These silicones may be chosen, for example, from polyalkylsiloxanes, among which non-limiting mention may be made of polydimethylsiloxanes containing trimethylsilyl end groups and having a viscosity of from 5×10⁻⁶ to 2.5 m²/s at 25° C., such as 1×10⁻⁵ to 1 m²/s. The viscosity of the silicones may be measured, for example, at 25° C. according to ASTM standard 445 Appendix C.

Among these polyalkylsiloxanes, non-limiting mention may be made of the following commercial products:

the Silbione oils of the 47 and 70 047 series or the Mirasil oils sold by Rhodia Chimie, such as, for example, the oil 70 047 V 500 000;

the oils of the Mirasil series sold by the company Rhodia Chimie;

the oils of the 200 series from the company Dow Corning, such as DC200 with a viscosity of 60 000 cSt;

the Viscasil oils from General Electric and certain oils of the SF series (SF 96, SF 18) from General Electric.

Non-limiting mention may also be made of polydimethylsiloxanes containing dimethylsilanol end groups (Dimethiconol according to the CTFA name) such as the oils of the 48 series from the company Rhodia Chimie.

In this category of polyalkylsiloxanes, non-limiting mention may also be made of the products sold under the names ABIL WAX 9800 and 9801 by the company Goldschmidt, which are poly(C₁-C₂₀)alkylsiloxanes.

The polyalkylarylsiloxanes may be chosen, for example, from linear and/or branched polydimethylmethylphenylsiloxanes and polydimethyldiphenylsiloxanes with a viscosity of from 1×10⁻⁵ to 5×10⁻² m²/s at 25° C.

Among these polyalkylarylsiloxanes, non-limiting mention may be made of the products sold under the following names:

the Silbione oils of the 70 641 series from Rhodia Chimie;

the oils of the Rhodorsil 70 633 and 763 series from Rhodia Chimie;

the oil Dow Corning 556 Cosmetic Grade Fluid from Dow Corning;

the silicones of the PK series from Bayer, such as the product PK20;

the silicones of the PN and PH series from Bayer, such as the products PN1000 and PH1000; and

certain oils of the SF series from General Electric, such as SF 1023, SF 1154, SF 1250 and SF 1265.

Non-limiting examples of silicone gums that can be used in accordance with the present disclosure include polydiorganosiloxanes having a number-average molecular masses ranging from 200 000 and 1 000 000, either alone or as a mixture in a solvent. This solvent may be chosen from volatile silicones, polydimethylsiloxane (PDMS) oils, polyphenylmethylsiloxane (PPMS) oils, isoparaffins, polyisobutylenes, methylene chloride, pentane, dodecane and tridecane, or mixtures thereof.

Non-limiting mention may be made of the following products:

polydimethylsiloxane,

polydimethylsiloxane/methylvinylsiloxane gums,

polydimethylsiloxane/diphenylsiloxane,

polydimethylsiloxane/phenylmethylsiloxane, and

polydimethylsiloxane/diphenylsiloxane/methylvinylsiloxane.

Additional non-limiting examples of products that can be used in accordance with the present disclosure include mixtures such as:

mixtures formed from a polydimethylsiloxane hydroxylated at the chain end (referred to as dimethiconol according to the nomenclature in the CTFA dictionary) and from a cyclic polydimethylsiloxane (referred to as cyclomethicone according to the nomenclature in the CTFA dictionary), such as the product Q2 1401 sold by the company Dow Corning;

mixtures formed from a polydimethylsiloxane gum with a cyclic silicone, such as the product SF 1214 Silicone Fluid from the company General Electric; this product is an SF 30 gum corresponding to a dimethicone, having a number-average molecular weight of 500 000, dissolved in the oil SF 1202 Silicone Fluid corresponding to decamethylcyclopentasiloxane;

mixtures of two PDMSs with different viscosities, e.g., of a PDMS gum and a PDMS oil, such as the product SF 1236 from the company General Electric. The product SF 1236 is a mixture of an SE 30 gum defined above, having a viscosity of 20 m²/s, and an SF 96 oil, with a viscosity of 5×10⁻⁶ m²/s. This product may contain, for example, 15% SE 30 gum and 85% SF 96 oil.

Non-limiting examples of organopolysiloxane resins that can be used in accordance with the present disclosure include crosslinked siloxane systems containing the following units:

R₂SiO_{2/2}, R₃SiO_{1/2}, RSiO_{3/2} and SiO_{4/2}, wherein R is chosen from a hydrocarbon-based group containing 1 to 16 carbon atoms or a phenyl group. Non-limiting examples of these products include those in which R is chosen from a C₁-C₄ lower alkyl radical, such as a methyl or a phenyl radical.

Among these resins, non-limiting mention may be made of the product sold under the name Dow Corning 593 or those sold under the names Silicone Fluid SS 4230 and SS 4267 by the company General Electric, which are silicones of dimethyl/trimethyl siloxane structure.

Non-limiting mention may also be made of the trimethyl siloxysilicate type resins sold under the names X22-4914, X21-5034 and X21-5037 by the company Shin-Etsu.

The organomodified silicones that may be used in accordance with the present disclosure include, for example, silicones as defined above and comprising in their structure at least one organofunctional group attached via a hydrocarbon-based radical.

Among the organomodified silicones, non-limiting mention may be made of polyorganosiloxanes comprising:

polyethyleneoxy and/or polypropyleneoxy groups optionally comprising C₆-C₂₄ alkyl groups, such as the products known as dimethicone copolyol sold by the company Dow Corning under the name DC 1248 or the oils Silwet L 722, L 7500, L 77 and L 711 by the company Union Carbide, and the (C₁₂)-alkylmethicone copolyol sold by the company Dow Corning under the name Q2 5200;

substituted or unsubstituted amine groups, such as the products sold under the name GP 4 Silicone Fluid and GP 7100 by the company Genesee, or the products sold under the names Q2 8220 and Dow Corning 929 or 939 by the company Dow Corning. The substituted amine groups are, in particular, C₁-C₄ aminoalkyl groups;

thiol groups such as the products sold under the names GP 72 A and GP 71 from Genesee;

alkoxylated groups such as the product sold under the name Silicone Copolymer F-755 by SWS Silicones and ABIL WAX 2428, 2434 and 2440 by the company Goldschmidt;

hydroxylated groups such as the polyorganosiloxanes containing a hydroxyalkyl function, described in French Patent FR 2,589,476, and corresponding to formula (XI):

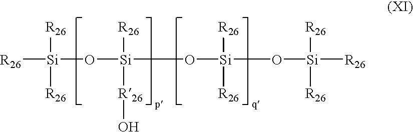

wherein the radicals R₂₆, which may be identical or different, are independently chosen from methyl and phenyl radicals; at least 60 mol % of the radicals R₂₆ are methyl radicals; the radical R′₂₆ is a C₂-C₁₈ divalent hydrocarbon-based alkylene chain unit; p′ ranges from 1 to 30 inclusive; and q′ ranges from 1 to 150 inclusive;

acyloxyalkyl groups such as, for example, the polyorganosiloxanes described in U.S. Pat. No. 4,957,732 and corresponding to formula (XII):

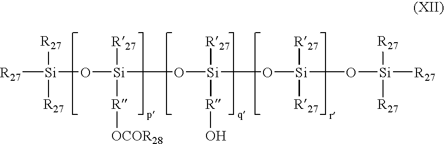

in which:
R₂₇, which may be the same or different, is independently chosen from a methyl, phenyl, —OCOR₂₈ or hydroxyl group, one of the radicals R₂₇ per silicon atom optionally being OH;
R′₂₇ is chosen from methyl or phenyl; at least 60 mol % of all the radicals R₄ and R′₄ denoting methyl;
R₂₈ is chosen from C₈-C₂₀ alkyls or alkenyls;
R″ is chosen from C₂-C₁₈ linear or branched divalent hydrocarbon-based alkylene radicals;
r′ ranges from 1 and 120 inclusive;
p′ ranges from 1 and 30;
q′ ranges from 0 to less than 0.5 p′, with the sum p′+q′ ranging from 1 to 30; the polyorganosiloxanes of formula (XII) may contain groups:

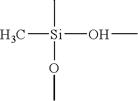

in an amount ranging from less than or equal to 15% of the sum p′+q′+r′;

anionic groups of carboxylic type, such as, for example, those in the products described in European Patent No. EP 186 507 from the company Chisso Corporation, or of alkylcarboxylic type, such as those present in the product X-22-3701E from the company Shin-Etsu; 2-hydroxyalkyl sulphonate; 2-hydroxyalkyl thiosulphate such as the products sold by the company Goldschmidt under the names ABIL S201 and ABIL S255;

hydroxyacylamino groups, such as the polyorganosiloxanes described in European Patent Application EP 342 834, such as, for example, the product Q2-8413 from the company Dow Corning.

According to the present disclosure, silicones comprising a polysiloxane portion and a portion consisting of a non-silicone organic chain may also be used, wherein one of the two portions constitutes the main chain of the polymer, and the other is grafted onto the main chain. Non-limiting examples of these polymers are described, for example, in European Patent Applications EP-A-412 704, EP-A-412 707, EP-A-640 105, and EP-A-582 152, International Application Nos. WO 95/00578 and WO 93/23009 and U.S. Pat. Nos. 4,693,935, 4,728,571 and 4,972,037. These polymers may, for example, be anionic or nonionic.

Such polymers may, for example, be copolymers obtained by free-radical polymerization of a monomer mixture comprising of:

a) 50 to 90% by weight of tert-butyl acrylate;

b) 0 to 40% by weight of acrylic acid; and

c) 5 to 40% by weight of silicone macromer of formula:

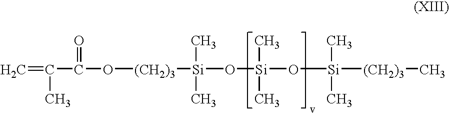

wherein v is a number ranging from 5 to 700; the weight percentages being calculated relative to the total weight of the monomers.

Other non-limiting examples of grafted silicone polymers include, for example, polydimethylsiloxanes (PDMS) onto which are grafted, via a connecting chain unit of thiopropylene type, mixed polymer units of poly(meth)acrylic acid type and of polyalkyl (meth)acrylate type and polydimethylsiloxanes (PDMS) onto which are grafted, via a connecting chain unit of thiopropylene type, polymer units of polyisobutyl (meth)acrylate type.

According to the present disclosure, all of the silicones may also be used in the form of an emulsion, nanoemulsion, or microemulsion.

Of the polyorganosiloxanes that may be used in accordance with the present disclosure, non-limiting mention may be made of:

non-volatile silicones chosen from the family of polyalkylsiloxanes containing trimethylsilyl end groups, such as oils having a viscosity ranging from 0.2 to 2.5 m²/s at 25° C., oils of the DC200 series from Dow Corning, including those with a viscosity of 60 000 cSt, of the SILBIONE 70047 and 47 series, and of the oil 70 047 V 500 000, which are sold by the company Rhodia Chimie, polyalkylsiloxanes containing dimethylsilanol end groups, such as dimethiconols, and polyalkylarylsiloxanes such as the oil SILBIONE 70641 V 200 sold by the company Rhodia Chimie;

the organopolysiloxane resin sold under the name Dow Corning 593; and

polysiloxanes containing amine groups, such as amodimethicones or trimethylsilylamodimethicones.

The cationic proteins or cationic protein hydrolysates are, for example, chemically modified polypeptides bearing quaternary ammonium groups at the end of the chain or grafted thereto. Their molecular mass may range, for example, from 1500 to 10 000, for example, from 2000 to 5000. Among these compounds, non-limiting mention may be made of:

collagen hydrolysates bearing triethylammonium groups, such as the products sold under the name QUAT-PRO E by the company Maybrook and referred to in the CTFA dictionary as “Triethonium Hydrolyzed Collagen Ethosulfate”;

collagen hydrolysates bearing trimethylammonium and trimethylstearylammonium chloride groups, sold under the name QUAT-PRO S by the company Maybrook and referred to in the CTFA dictionary as “Steartrimonium Hydrolyzed Collagen”;

animal protein hydrolysates bearing trimethylbenzylammonium groups such as the products sold under the name CROTEIN BTA by the company Croda and referred to in the CTFA dictionary as “Benzyltrimonium hydrolyzed animal protein”;

protein hydrolysates bearing, on the polypeptide chain, quaternary ammonium groups containing at least one alkyl radical having from 1 to 18 carbon atoms.

Among these protein hydrolysates, non-limiting mention may be made of:

CROQUAT L, in which the quaternary ammonium groups contain a C₁₂ alkyl group;

CROQUAT M, in which the quaternary ammonium groups contain C₁₀-C₁₈ alkyl groups;

CROQUAT S, in which the quaternary ammonium groups contain a C₁₈ alkyl group; and

CROTEIN Q, in which the quaternary ammonium groups contain at least one alkyl group having from 1 to 18 carbon atoms.

These various products are sold by the company Croda.

Other quaternized proteins or hydrolysates include, for example, those corresponding to formula (XIV):

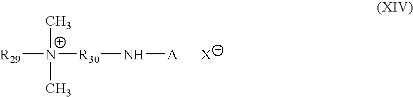

wherein:

X⁻ is chosen from anions of an organic or mineral acid;

A is chosen from protein residues derived from hydrolysates of collagen protein;

R₂₉ is chosen from lipophilic groups containing up to 30 carbon atoms; and

R₃₀ is chosen from an alkylene group having 1 to 6 carbon atoms.

Of these compounds, non-limiting mention may be made, for example, of the products sold by the company Inolex under the name LEXEIN QX 3000, referred to in the CTFA dictionary as “Cocotrimonium Collagen Hydrolysate”.

Non-limiting mention may also be made of quaternized plant proteins such as wheat, corn or soybean proteins. As examples of quaternized wheat proteins, non-limiting mention may be made of those sold by the company Croda under the names HYDROTRITICUM WQ or QM, referred to in the CTFA dictionary as “Cocodimonium Hydrolysed Wheat Protein”, HYDROTRITICUM QL, referred to in the CTFA dictionary as “Laurdimonium Hydrolysed Wheat Protein” and HYDROTRITICUM QS, referred to in the CTFA dictionary as “Steardimonium Hydrolysed Wheat Protein.”

As used herein, the compounds of ceramide type include, for example, natural or synthetic ceramides and/or glycoceramides and/or pseudoceramides and/or neoceramides.

Non-limiting examples of compounds of ceramide type are described in German Patent Application Nos. DE 4 424 530, DE 4 424 533, DE 4 402 929, and DE 4 420 736, International Published Application Nos. WO 95/23807, WO 94/07844, WO 95/16665, WO 94/07844, WO 94/24097 and WO 94/10131, French Application No. FR-2 673 179, and European Application Nos. EP-A-0 646 572 and EP-A-0 227 994, the contents of all of which are incorporated herein by reference.

Non-limiting examples of compounds of ceramide type that may be used in accordance with the present disclosure include:
2-N-linoleoylaminooctadecane-1,3-diol,
2-N-oleoylaminooctadecane-1,3-diol,
2-N-palmitoylaminooctadecane-1,3-diol,
2-N-stearoylaminooctadecane-1,3-diol,
2-N-behenoylaminooctadecane-1,3-diol,
2-N-[2-hydroxypalmitoyl]aminooctadecane-1,3-diol,
2-N-stearoylaminooctadecane-1,3,4-triol, e.g., N-stearoylphytosphingosine,
2-N-palmitoylaminohexadecane-1,3-diol,
bis(N-hydroxyethyl-N-cetyl)malonamide,
N-(2-hydroxyethyl)-N-(3-cetyloxy-2-hydroxypropyl)cetylamide,
N-docosanoyl-N-methyl-D-glucamine,
and mixtures thereof.

Cationic surfactants may also be used. Non-limiting examples of suitable cationic surfactants include: optionally polyoxyalkylenated primary, secondary or tertiary fatty amine salts; quaternary ammonium salts; imidazoline derivatives; and amine oxides of cationic nature.

Non-limiting examples of quaternary ammonium salts include:

those of formula (XV) below:

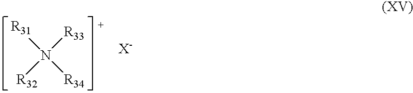

wherein the radicals R₃₁ to R₃₄, which may be identical or different, are independently chosen from linear or branched aliphatic radicals containing from 1 to 30 carbon atoms, or aromatic radicals such as aryl or alkylaryl. The aliphatic radicals may comprise heteroatoms such as, for example, oxygen, nitrogen, sulphur or halogens. The aliphatic radicals are chosen, for example, from alkyl, alkoxy, polyoxy(C₂-C₆)alkylene, alkylamide, (C₁₂-C₂₂)alkylamido(C₂-C₆)alkyl, (C₁₂-C₂₂)alkylacetate and hydroxyalkyl radicals, comprising from about 1 to 30 carbon atoms; X⁻ is an anion chosen from the group of halides, phosphates, acetates, lactates, (C₂-C₆)alkyl sulphates and alkyl or alkylaryl sulphonates;

quaternary ammonium salts of imidazolinium, such as, for example, the salt of formula (XVI) below:

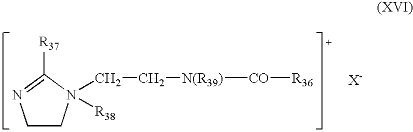

wherein R₃₆ is chosen from alkenyl or alkyl radicals containing from 8 to 30 carbon atoms, for example tallow fatty acid derivatives, R₃₇ is chosen from a hydrogen atom, a C₁-C₄ alkyl radical or an alkenyl or alkyl radical containing from 8 to 30 carbon atoms, R₃₈ is chosen from a C₁-C₄ alkyl radical, R₃₉ is chosen from a hydrogen atom or a C₁-C₄ alkyl radical, and X⁻ is an anion chosen from the group of halides, phosphates, acetates, lactates, alkyl sulphates and alkyl or alkylaryl sulphonates.

In a non-limiting embodiment, R₃₆ and R₃₇ represent a mixture of alkenyl or alkyl radicals containing from 12 to 21 carbon atoms, for example tallow fatty acid derivatives, R₃₈ is a methyl radical, and R₃₉ is a hydrogen atom. Such a product is sold, for example, under the name “Rewoquat W 75” by the company Degussa;

diquaternary ammonium salts of formula (XVII):

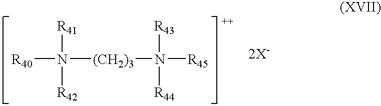

wherein R₄₀ is chosen from aliphatic radicals containing from 16 to 30 carbon atoms, R₄₁, R₄₂, R₄₃, R₄₄ and R₄₅, which may be identical or different, are each independently chosen from hydrogen or an alkyl radical containing from 1 to 4 carbon atoms, and X⁻ is an anion chosen from halides, acetates, phosphates, nitrates and methyl sulphates. Such diquaternary ammonium salts may, for example, comprise propane tallow diammonium dichloride;

quaternary ammonium salts containing at least one ester function.

Non-limiting examples of quaternary ammonium salts containing at least one ester function that may be used according to the present disclosure include, for example, those of formula (XVIII) below:

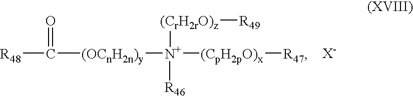

wherein:

R₄₆ is chosen from C₁-C₆ alkyl radicals and C₁-C₆ hydroxyalkyl or dihydroxyalkyl radicals;

R₄₇ is chosen from:
a radical

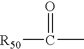

linear or branched, saturated or unsaturated C₁-C₂₂ hydrocarbon-based radicals R₅₁, and
a hydrogen atom,

R₄₉ is chosen from:
a radical

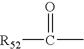

linear or branched, saturated or unsaturated C₁-C₆ hydrocarbon-based radicals R₅₃, and
a hydrogen atom,

R₄₈, R₅₀ and R₅₂, which may be identical or different, are independently chosen from linear or branched, saturated or unsaturated C₇-C₂₁ hydrocarbon-based radicals;

n, p and r, which may be identical or different, are independently chosen from integers ranging from 2 to 6;

y is an integer ranging from 1 to 10;

x and z, which may be identical or different, are independently chosen from integers ranging from 0 to 10;

X⁻ is chosen from simple or complex, organic or inorganic anions; with the proviso that the sum x+y+z ranges from 1 to 15, that when x is 0, R₄₇ is chosen from linear or branched, saturated or unsaturated C₁-C₂₂ hydrocarbon-based radicals R₅₁, and that when z is 0, R₄₉ is chosen from linear or branched, saturated or unsaturated C₁-C₆ hydrocarbon-based radicals R₅₃.

The R₄₆ alkyl radicals may be linear or branched, and in at least one embodiment, are linear.

R₄₆ may, for example, be chosen from a methyl, ethyl, hydroxyethyl or dihydroxypropyl radical and in at least one embodiment, a methyl or ethyl radical.

The sum x+y+z may also range, for example, from 1 to 10.

When R₄₇ is a hydrocarbon-based radical R₅₁, it may, for example, be long and contain from 12 to 22 carbon atoms, or short and contain from 1 to 3 carbon atoms.

When R₄₉ is a hydrocarbon-based radical R₅₃, it may, for example, contain from 1 to 3 carbon atoms.

R₄₈, R₅₀ and R₅₂, which may be identical or different, may, for example, be chosen from linear or branched, saturated or unsaturated C₁₁-C₂₁ hydrocarbon-based radicals, and more particularly from linear or branched, saturated or unsaturated, C₁₁-C₂₁ alkyl and alkenyl radicals.

x and z, which may be identical or different, may, for example, be chosen from 0 or 1.

In at least one non-limiting embodiment, y is equal to 1.

n, p and r, which may be identical or different, may for example, be chosen from 2 or 3. In a non-limiting embodiment, n p, and r are equal to 2.

The anion X⁻ may be chosen, for example, from halides (e.g., chloride, bromide or iodide) and alkyl sulphates, such as methyl sulphate. However, methanesulphonate, phosphate, nitrate, tosylate, an anion derived from an organic acid, such as acetate or lactate, or any other anion that is compatible with the ammonium containing an ester function, may also be used.

In a non-limiting embodiment, the anion X⁻ is chosen from chloride and methyl sulphate.

Non-limiting examples of ammonium salts include those of formula (XVIII), in which:

R₄₆ is chosen from a methyl or ethyl radical,

x and y are equal to 1;

z is equal to 0 or 1;

n, p and r are equal to 2;

R₄₇ is chosen from:
a radical

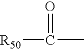

methyl, ethyl or C₁₄-C₂₂ hydrocarbon-based radicals; and
a hydrogen atom;

R₄₉ is chosen from:
a radical

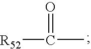

and
a hydrogen atom;

R₄₈, R₅₀ and R₅₂, which may be identical or different, are independently chosen from linear or branched, saturated or unsaturated C₁₃-C₁₇ hydrocarbon-based radicals, such as linear or branched, saturated or unsaturated C₁₃-C₁₇ alkyl and alkenyl radicals.

In at least one non-limiting embodiment, the hydrocarbon-based radicals are linear.

Non-limiting examples that may be mentioned include the compounds of formula (XVI), such as the diacyloxyethyldimethylammonium, diacyloxyethylhydroxyethylmethylammonium, monoacyl-oxyethyldihydroxyethylmethylammonium, triacyloxyethylmethylammonium and monoacyloxyethylhydroxyethyldimethylammonium salts (chloride or methyl sulphate in particular), and mixtures thereof. The acyl radicals may, for example, contain 14 to 18 carbon atoms and may be obtained, for example, from a plant oil such as palm oil or sunflower oil. When the compound contains several acyl radicals, these radicals may be identical or different.

These products may be obtained, for example, by direct esterification of triethanolamine, triisopropanolamine, an alkyldiethanolamine or an alkyldiisopropanolamine, which are optionally oxyalkylenated, with fatty acids or with fatty acid mixtures of plant or animal origin, or by transesterification of the methyl esters thereof. This esterification is followed by a quaternization using an alkylating agent such as an alkyl halide (e.g., a methyl or ethyl halide), a dialkyl sulphate (e.g., dimethyl or diethyl sulphate), methyl methanesulphonate, methyl para-toluenesulphonate, glycol chlorohydrin or glycerol chlorohydrin.

Such compounds are sold, for example, under the names DEHYQUART by the company Cognis, STEPANQUAT by the company Stepan, NOXAMIUM by the company CECA or REWOQUAT WE 18 by the company Degussa.

It is also possible to use ammonium salts containing at least one ester function, such as those described in U.S. Pat. Nos. 4,874,554 and 4,137,180.

Among the quaternary ammonium salts of formula (XV), non-limiting mention may be made of tetraalkylammonium chlorides such as, for example, dialkyldimethylammonium chlorides or alkyltrimethylammonium chlorides, in which the alkyl radical contains from about 12 to 22 carbon atoms. Further non-limiting mention may be made of behenyltrimethylammonium chloride, distearyldimethylammonium chloride, cetyltrimethylammonium chloride, or benzyldimethylstearylammonium chloride, or, on the other hand, stearamidopropyldimethyl(myristyl acetate)ammonium chloride sold under the name CERAPHYL 70 by the company Van Dyk.

Of the saturated fatty acids that may be used, non-limiting mention is made of myristic acid, palmitic acid, stearic acid, behenic acid and isostearic acid.

Of the fatty acid esters that may be used, non-limiting mention is made of carboxylic acid esters, such as mono-, di-, tri- or tetracarboxylic esters.

The monocarboxylic acid esters are different from those of the present disclosure and are, for example, linear or branched, saturated or unsaturated C₁-C₂₆ aliphatic acid monoesters and linear and branched, saturated or unsaturated, C₁-C₂₆ aliphatic alcohols, the total carbon number of these esters being greater than or equal to 10.

Among the monoesters, non-limiting mention may be made of dihydroabietyl behenate; octyldodecyl behenate; isocetyl behenate; cetyl lactate; C₁₂-C₁₅ alkyl lactate; isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methylacetyl ricinoleate; myristyl stearate; octyl isononanoate; 2-ethylhexyl isononate; octyl palmitate; octyl pelargonate; octyl stearate; octyidodecyl erucate; oleyl erucate; ethyl and isopropyl palmitates, 2-ethylhexyl palmitate, 2-octyidecyl palmitate, alkyl myristates such as isopropyl, butyl, cetyl or 2-octyidodecyl myristate, hexyl stearate, butyl stearate, isobutyl stearate; dioctyl malate, hexyl laurate, 2-hexyldecyl laurate, isostearyl neopentanoate, and isodecyl neopentanoate.

C₄-C₂₂ di- or tricarboxylic acid esters of C₁-C₂₂ alcohols and mono-, di- or tricarboxylic acid esters of C₂-C₂₆ di-, tri-, tetra- or pentahydroxy alcohols may also be used.

Non-limiting mention may also be made of: diethyl sebacate; diisopropyl sebacate; diisopropyl adipate; di-n-propyl adipate; dioctyl adipate; diisostearyl adipate; dioctyl maleate; glyceryl undecylenate; octyidodecylstearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate dicaprate; tridecyl erucate; triisopropyl citrate; triisostearyl citrate; glyceryl trilactate; glyceryl trioctanoate; trioctyidodecyl citrate; and trioleyl citrate.

Among the esters mentioned above, further non-limiting mention may be made of ethyl and isopropyl palmitates, 2-ethylhexyl palmitate, 2-octyldecyl palmitate, alkyl myristates such as isopropyl, butyl, cetyl or 2-octyldodecyl myristate, hexyl stearate, butyl stearate, isobutyl stearate; dioctyl malate, hexyl laurate, 2-hexyldecyl laurate, isononyl isononanoate, cetyl octanoate, isostearyl neopentanoate, and isodecyl neopentanoate.

As examples of the fluoro oils that may be used, non-limiting mention may be made of the perfluoropolyethers described in European Patent Application No. EP-A-486 135, and the fluorohydrocarbon compounds described in International Patent Application No. WO 93/11103. The content of these two patent applications is incorporated herein by reference.

As used herein, the term, “fluorohydrocarbon compounds,” means compounds whose chemical structure contains a carbon skeleton in which certain hydrogen atoms have been replaced with fluorine atoms.

The fluoro oils may also be fluorocarbons such as fluoroamines, such as perfluorotributylamine, fluorohydrocarbons, such as perfluorodecahydronaphthalene, fluoro esters, and fluoro ethers.

Non-limiting examples of such perfluoropolyethers include those that are sold under the trade names Fomblin by the company Montefluos and Krytox by the company Du Pont.

Among the fluorohydrocarbon compounds, non-limiting mention may be made of fluorine-containing fatty acid esters, such as the product sold under the name NOFABLE FO by the company Nippon Oil.

It is of course possible to use mixtures of conditioning agents.

According to the present disclosure, the conditioning agent(s) may be present in an amount ranging from 0.001% to 20% by weight, such as from 0.01% to 10% by weight, for example from 0.1% to 3% by weight, relative to the total weight of the composition (B) or (C).

The compositions (B) and (C) for protecting the color of keratin fibers according to the present disclosure may, for example, be in the form of an aqueous or aqueous-alcoholic lotion. The cosmetic compositions according to the present disclosure may also be in the form of an oil, a gel, a milk, a cream, an emulsion or a mousse.

The compositions (B) and (C) for protecting the color of keratin fibers may be packaged in various forms, such as in vaporizers, pump-dispenser bottles or in aerosol containers in order to apply the composition in vaporized form or in the form of a mousse. Such packaging forms are useful, for example, when it is desired to obtain a spray, a lacquer or a mousse for treating the hair.

The pH of the composition for protecting keratin fibers may range, for example, from 1 to 11. The pH may be adjusted to any desired range, for example, from 2 to 6, by means of acidifying or basifying agents that are well known in the prior art for compositions applied to keratin fibers.

Among the basifying agents, non-limiting mention may be made of aqueous ammonia, alkali metal carbonates, alkanolamines such as monoethanolamine, diethanolamine and triethanolamine and also derivatives thereof, oxyethylenated and/or oxypropylenated hydroxyalkylamines and ethylenediamines, sodium hydroxide, potassium hydroxide and compounds having the formula below:

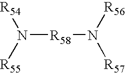

wherein R₅₈ is a propylene residue optionally substituted with a hydroxyl group or a C₁-C₄ alkyl radical; and R₅₄, R₅₅, R₅₆ and R₅₇, which may be identical or different, are independently chosen from a hydrogen atom, a C₁-C₄ alkyl radical or a C₁-C₄ hydroxyalkyl radical.

The acidifying agents may, for example, be those that are conventionally known, for example, mineral or organic acids such as hydrochloric acid or orthophosphoric acid, carboxylic acids such as tartaric acid, citric acid or lactic acid, or sulphonic acid.

A non-limiting aspect of the present disclosure is a process for protecting the color of artificially dyed keratin fibers with respect to washing or atmospheric agents, comprising applying at least:

a) a composition (B) comprising, in a cosmetically acceptable medium, at least one agent for protection against the effects of atmospheric agents, such as light;

b) at least one composition (C) comprising, in a cosmetically acceptable medium, at least one zinc-based compound;

wherein the at least one composition (C) may be applied before or after dyeing of the artificially dyed keratin fibers, and said composition (B) is applied after dyeing of the artificially dyed keratin fibers.

In one non-limiting embodiment, the at least one composition (C) is applied after dyeing of said artificially dyed keratin fibers, and before or after the application of the composition (B). In a further non-limiting embodiment, said at least one composition (C) is applied after application of the composition (B).

The process according to the present disclosure may also comprise at least one step of rinsing and/or a step of washing with shampoo before or after the application of the composition (B) or (C).

The process according to the present disclosure may further comprise at least one additional step of total or partial drying of the keratin fibers, e.g., with a hairdryer, for each application of compositions (B) and (C).

The process for protecting the color of keratin fibers may further comprise at least one step of heating the composition (B) and/or the composition (C), which will then be applied directly to the keratin fibers. In at least one embodiment, the heating temperature is less than 120° C.

According to a specific form of the present disclosure, the process for protecting the color of keratin fibers may comprise a step of heating the keratin fibers after or during the application of the composition (B) and/or of the composition (C).

The heating of the keratin fibers may, for example, be carried out using an iron, a liquid water/steam mixture, or by means of a heating hood.

Heating irons that may be used in the context of the present disclosure include those conventionally used in the haircare field. Such irons include, for example, a crimping iron or a smoothing iron, and are well known in the field of hair treatment. Additional examples of irons that can be used for implementing the present disclosure include flat or round irons, such as those described in U.S. Pat. Nos. 4,103,145, 4,308,878, 5,983,903, 5,957,140 and 5,494,058. The iron may be applied by successive separate touches of a few seconds, or by gradually moving or sliding it along the locks. A pause may be present between the application of the color-protecting composition and the application of the heating iron to the keratin fibers. This pause may, for example, range from 30 seconds to 60 minutes, such as from 1 to 30 minutes. The temperature imparted by said heating may, for example, range from 60° C. to 120° C.

The liquid water/steam mixture that can be used in the context of the present disclosure may, for example, have a temperature of at least 35° C.

The liquid water/steam mixture may constitute a mist. The mixture may also contain at least one other gas such as oxygen or nitrogen, mixtures of gases such as air, or other vaporizable compounds.

The temperature of the liquid water/steam mixture may, for example, range from greater than or equal to 40° C., such as from 40° C. to 75° C.

The liquid water/steam mixture may, for example, be brought into contact with the fiber for a period of time ranging from 1 second to 1 hour, such as from 5 minutes to 15 minutes. Of course, the application of said mixture may be repeated several times on the same fiber, with each operation being performed for a period of time as indicated above. For example, the composition containing the zinc-based compounds is first applied to the hair and these locks thus impregnated, are then subjected to the action of the liquid water/steam mixture under the conditions mentioned above, and the locks thus treated are then cooled, for example by sending over or through them a stream of cold air or of air at ambient temperature.

The liquid water/steam mixture used according to the present disclosure may be produced using any apparatus known per se and intended for this purpose. For example, an apparatus comprising at least one steam generator directly connected to a hood that diffuses the liquid water/steam mixture onto the keratin fibers, e.g., human hair, may be used. As an example of such an apparatus, non-limiting mention is made of the apparatus sold under the name Micromist® by the company Takara Belmont.

The present disclosure also relates to a process for dyeing keratin fibers, including human keratin fibers such as the hair, comprising applying to the fibers at least:

(i) a direct dye composition (A) or an oxidation dye composition (A) in the presence of an oxidizing agent for a period of time sufficient to develop a desired color,

(ii) a composition (B) comprising, in a cosmetically acceptable medium, at least one agent for protection against the effects of atmospheric agents, such as light,

(iii) a composition (C) containing, in a cosmetically acceptable medium, at least one zinc-based mineral compound and/or a zinc-based non-nitrogenous organic compound; wherein the composition (C) may be applied before or after application of the direct dye composition (A) or oxidation dye composition (A), and the composition (B) is applied after application of the direct dye composition (A) or oxidation dye composition (A).

In one non-limiting embodiment, the composition (C) comprising the zinc-based compound is applied to the fibers after application of the direct dye composition (A) or oxidation dye composition (A), and before or after the application of the composition (B). In a further non-limiting embodiment, composition (C) is applied after composition (B).

The application of the composition (A) may be followed by rinsing and/or drying of the keratin fibers.

The application of composition (B) and/or of composition (C) may be followed by rinsing and/or drying of the keratin fibers. The composition (B) and/or the composition (C) may also be preheated under the same conditions defined above. The application of the composition (B) and/or of the composition (C) may also be followed by heating of the keratin fibers under the same conditions defined above.

In one non-limiting embodiment, composition (C) is applied after the direct dye or oxidation dye composition (A). The compositions (B) and/or (C) may be applied immediately after dyeing or after a delay. As used herein, the term, “after a delay,” means an application that takes place a few hours or one or several days (from 1 to 15 days) after dyeing. For example, the composition (B) and/or the composition (C) may be applied immediately after dyeing the keratin fibers. The application of the compositions may also be repeated between two colorings.

The nature and concentration of the dyes present in the dye composition (A) is not critical.

In the case of lightening direct dyeing operations, the dye compositions (A) may result from the mixing, at the time of use, of a dye composition (A₁) containing at least one direct dye and of a composition (A₂) containing an oxidizing agent.

In the case of oxidation dyeing, the dye compositions (A) may result from the mixing, at the time of use, of a dye composition (A₃) containing at least one oxidation base and, optionally, at least one coupler and/or a direct dye, and of a composition (A₄) containing an oxidizing agent.

As examples of direct dyes that may be used in accordance with the present disclosure, non-limiting mention may be made of compounds that absorb light radiation in the visible range (400-750 nm). They may, for example, be of nonionic, anionic or cationic nature.

In generally, the direct dyes may be chosen from nitrobenzene dyes and azo, anthraquinone, naphthoquinone, benzoquinone, phenothiazine, indigoid, xanthene, phenanthridine, phthalocyanin and triarylmethane-based dyes, alone or as mixtures.

Among the nitrobenzene dyes, non-limiting mention may be made of the following red or orange compounds: 1-hydroxy-3-nitro-4-N-(γ-hydroxypropyl)aminobenzene, N-(β-hydroxyethyl)amino-3-nitro-4-aminobenzene, 1-amino-3-methyl-4-N-(β-hydroxyethyl)amino-6-nitrobenzene, 1-hydroxy-3-nitro-4-N-(β-hydroxyethyl)amino-benzene, 1,4-diamino-2-nitrobenzene, 1-amino-2-nitro-4-methylaminobenzene, N-(β-hydroxyethyl)-2-nitro-para-phenylenediamine, 1-amino-2-nitro-4-(β-hydroxyethyl)-amino-5-chlorobenzene, 2-nitro-4-aminodiphenylamine, 1-amino-3-nitro-6-hydroxybenzene, 1-(β-aminoethyl)amino-2-nitro-4-(β-hydroxyethyloxy)benzene, 1-(β,γ-dihydroxypropyl)oxy-3-nitro-4-(β-hydroxyethyl)aminobenzene, 1-hydroxy-3-nitro-4-aminobenzene, 1-hydroxy-2-amino-4,6-dinitrobenzene, 1-methoxy-3-nitro-4-(β-hydroxyethyl)aminobenzene, 2-nitro-4′-hydroxydiphenylamine, and 1-amino-2-nitro-4-hydroxy-5-methylbenzene, alone or as mixtures.

As example of nitrobenzene direct dyes, non-limiting mention may be made of dyes of yellow and green-yellow type, for instance 1-β-hydroxyethyloxy-3-methylamino-4-nitrobenzene, 1-methylamino-2-nitro-5-(β,γ-dihydroxypropyl)oxybenzene, 1-(β-hydroxyethyl)amino-2-methoxy-4-nitrobenzene, 1-(β-aminoethyl)amino-2-nitro-5-methoxybenzene, 1,3-bis(β-hydroxyethyl)amino-4-nitro-6-chlorobenzene, 1-amino-2-nitro-6-methylbenzene, 1-(β-hydroxyethyl)amino-2-hydroxy-4-nitrobenzene, N-(β-hydroxyethyl)-2-nitro-4-trifluoromethylaniline, 4-(β-hydroxyethyl)amino-3-nitrobenzenesulphonic acid, 4-ethylamino-3-nitrobenzoic acid, 4-(β-hydroxyethyl)amino-3-nitrochlorobenzene, 4-(β-hydroxyethyl)amino-3-nitromethylbenzene, 4-(β,γ-dihydroxypropyl)amino-3-nitrotrifluoromethylbenzene, 1-(β-ureidoethyl)amino-4-nitrobenzene, 1,3-diamino-4-nitrobenzene, 1-hydroxy-2-amino-5-nitrobenzene, 1-amino-2-[tris(hydroxymethyl)methyl]amino-5-nitrobenzene, 1-(β-hydroxyethyl)amino-2-nitrobenzene, and 4-(β-hydroxyethyl)amino-3-nitrobenzamide.

Blue or violet nitrobenzene dyes may also be used, including, for example, 1-(β-hydroxyethyl)amino-4-N,N-bis(β-hydroxyethyl)amino-2-nitrobenzene, 1-(γ-hydroxypropyl)amino-4-N,N-bis(p-hydroxyethyl)amino-2-nitrobenzene, 1-(β-hydroxy-ethyl)amino-4-(N-methyl, N-β-hydroxyethyl)amino-2-nitrobenzene, 1-(β-hydroxyethyl)amino-4-(N-ethyl, N-β-hydroxyethyl)amino-2-nitrobenzene, 1-(β,γ-dihydroxypropyl)amino-4-(N-ethyl, N-β-hydroxyethyl)amino-2-nitrobenzene, the 2-nitro-para-phenylenediamines of the following formula:

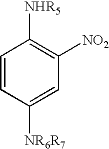

wherein:

R₆ is chosen from a C₁-C₄ alkyl radical or a β-hydroxyethyl, β-hydroxypropyl or γ-hydroxypropyl radical;

R₅ and R₇, which may be identical or different, are independently chosen from a β-hydroxyethyl, β-hydroxypropyl, γ-hydroxypropyl or β,γ-dihydroxypropyl radical, wherein at least one of the radicals R₆, R₇ or R₅ is a γ-hydroxypropyl radical, and R₆ and R₇ are not both a β-hydroxyethyl radical when R₆ is a γ-hydroxypropyl radical. Non-limiting examples of such compounds include those described French Patent No. FR 2 692 572.

As used herein, Azo dyes are compounds comprising in their structure at least one —N═N— sequence not included in a ring; methine dyes are compounds comprising in their structure at least one —C═C— sequence not included in a ring; and azomethine dyes are compounds comprising in their structure at least one —C═N— sequence not included in a ring.

The triarylmethane-based dyes comprise in their structure at least one sequence below:

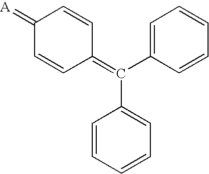

wherein A is an oxygen or nitrogen atom.

The xanthene dyes comprise in their structure at least one sequence of formula:

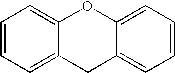

The phenanthridine dyes comprise in their structure at least one sequence of formula:

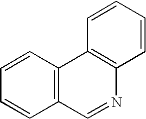

The phthalocyanine dyes comprise in their structure at least one sequence of formula:

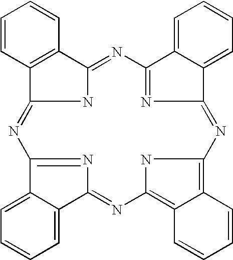

The phenothiazine dyes comprise in their structure at least one sequence below:

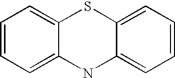

The direct dyes may be chosen, for example, from basic dyes such as those listed in the Color Index, 3rd edition, including those listed under the names Basic Brown 16, Basic Brown 17, Basic Yellow 57, Basic Red 76, Basic Violet 10, Basic Blue 26 and Basic Blue 99; from the acidic direct dyes such as those listed in the Color Index, 3rd edition, under the names Acid Orange 7, Acid Orange 24, Acid Yellow 36, Acid Red 33, Acid Red 184, Acid Black 2, Acid Violet 43, and Acid Blue 62; or cationic direct dyes, such as those described in International Published Patent Applications WO 95/01772 and WO 95/15144 and European Patent Application EP 714 954, for example Basic Red 51, Basic Orange 31 and Basic Yellow 87. The content of these documents is incorporated herein by reference.

When they are present, the direct dye may, for example, be present in an amount ranging from 0.0005% to 12% by weight, such as from 0.005% to 6% by weight, relative to the total weight of the composition.

The oxidation bases may be chosen, for example, from the oxidation bases conventionally used in oxidation dyeing. Of such oxidation bases, non-limiting mention may be made of para-phenylenediamines, bis(phenyl)alkylenediamines, para-aminophenols, ortho-aminophenols, and heterocyclic bases.

Among the para-phenylenediamines, non-limiting mention may be made of para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, N,N-diethyl-4-amino-3-methylaniline, N,N-bis(p-hydroxyethyl)-para-phenylenediamine, 4-N,N-bis(β-hydroxyethyl)amino-2-methylaniline, 4-N,N-bis(β-hydroxy-ethyl)amino-2-chloroaniline, 2-p-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxy-propyl)-para-phenylenediamine, N-(4′-aminophenyl)-para-phenylenediamine, N-phenyl-para-phenylenediamine, 2-β-hydroxyethyloxy-para-phenylenediamine, 2-β-acetyl-aminoethyloxy-para-phenylenediamine and N-(β-methoxyethyl)-para-phenylenediamine, and the acid addition salts thereof.

Among the para-phenylenediamines mentioned above, further non-limiting mention may be made of para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine, and the acid addition salts thereof.

Among the bis(phenyl)alkylenediamines, non-limiting mention may be made of N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-amino-phenyl)tetramethylenediamine, N,N′-bis(4-methylaminophenyl)tetramethylenediamine, N,N′-bis(ethyl)-N,N′-bis(4′-amino-3′-methylphenyl)ethylenediamine and 1,8-bis(2,5-diaminophenoxy)-3,5-dioxaoctane, and the addition salts thereof with an acid.

Among the para-aminophenols, non-limiting mention may be made of para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxy-ethylaminomethyl)phenol and 4-amino-2-fluorophenol, and the acid addition salts thereof.

Among the ortho-aminophenols, non-limiting mention may be made of 2-aminophenol, 2-amino-5-methylphenol, 2-amino-6-methylphenol and 5-acetamido-2-aminophenol, and the acid addition salts thereof.

Among the heterocyclic bases, non-limiting mention may be made of pyridine derivatives, pyrimidine derivatives and pyrazole derivatives.

Among the pyridine derivatives, non-limiting mention may be made of the compounds described, for example, in British Patent Nos. GB 1 026 978 and GB 1 153 196, as well as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine and 3,4-diaminopyridine, and the acid addition salts thereof.

Among the pyrimidine derivatives, non-limiting mention may be made of the compounds described, for example, in German Patent DE 2 359 399; Japanese Patents JP 2-19576; and JP 05-163 124; European Patent EP 0 770 375 and International Patent Application WO 96/15765, such as 2,4,5,6-tetraminopyrimidine, 4-hydroxy-2,5,6-triamino-pyrimidine, 2-hydroxy-4,5,6-triaminopyrimidine, 2,4-dihydroxy-5,6-diaminopyrimidine and 2,5,6-triaminopyrimidine, and pyrazolopyrimidine derivatives such as those mentioned in French Patent Application FR-A-2 750 048, for example pyrazolo[1,5-a]pyrimidine-3,7-diamine; 2,5-dimethylpyrazolo[1,5-a]pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-aminopyrazolo[1,5-a]pyrimidin-5-ol; 2-(3-aminopyrazolo[1,5-a]pyrimidin-7-ylamino)ethanol, 2-(7-aminopyrazolo[1,5-a]pyrimidin-3-ylamino)ethanol, 2-[(3-amino-pyrazolo[1,5-a]pyrimidin-7-yl)(2-hydroxyethyl)amino]ethanol, 2-[(7-aminopyrazolo[1,5-a]-pyrimidin-3-yl)(2-hydroxyethyl)amino]ethanol, 5,6-dimethylpyrazolo[1,5-a]pyrimidine-3,7-diamine, 2,6-dimethylpyrazolo[1,5-a]pyrimidine-3,7-diamine, 2,5,N7,N7-tetramethyl-pyrazolo[1,5-a]pyrimidine-3,7-diamine and 3-amino-5-methyl-7-imidazolylpropyl-aminopyrazolo[1,5-a]pyrimidine, the acid addition salts thereof, and the tautomeric forms thereof, when a tautomeric equilibrium exists.

Among the pyrazole derivatives, non-limiting mention may be made of the compounds described in German Patents DE 3 843 892 and DE 4 133 957 and International Patent Applications WO 94/08969 and WO 94/08970, French Application No. FR-A-2 733 749 and German Application No. DE 195 43 988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethyl-pyrazole, 3,4,5-triaminopyrazole, 1-methyl-3,4,5-triaminopyrazole, 3,5-diamino-1-methyl-4-methylaminopyrazole and 3,5-diamino-4-(β-hydroxyethyl)amino-1-methylpyrazole, and the acid addition salts thereof.

When they are used, these oxidation bases may be present in an amount ranging from 0.0005% to 12% by weight, such as from 0.005% to 6% by weight, relative to the total weight of the dye composition.

The oxidation dye compositions in accordance with the present disclosure may also contain at least one coupler and/or at least one direct dye, e.g., to modify the shades or to enrich them with tints.

The couplers that may be used in the oxidation dye compositions according to the present disclosure may be chosen, for example, from the couplers conventionally used in oxidation dyeing. Among such couplers, non-limiting mention may be made of meta-phenylenediamines, meta-aminophenols, meta-diphenols, naphthols and heterocyclic couplers, such as indole derivatives, indoline derivatives, pyridine derivatives, indazole derivatives, pyrazolo[1,5-b]-1,2,4-triazole derivatives, pyrazolo[3,2-c]-1,2,4-triazole derivatives, benzimidazole derivatives, benzothiazole derivatives, benzoxazole derivatives, 1,3-benzodioxole derivatives and pyrazolones, and the acid addition salts thereof.

Of such couplers, further non-limiting mention may be made of 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, sesamol, x-naphthol, 2-methyl-1-naphthol, 6-hydroxyindole, 4-hydroxyindole, 4-hydroxy-N-methyl-indole, 6-hydroxyindoline, 6-hydroxybenzomorpholine, 3,5-diamino-2,6-dimethoxypyridine, 1-N-(β-hydroxyethyl)amino-3,4-methylenedioxybenzene, 2,6-bis(β-hydroxy-ethyleneamino)toluene, 2,6-dihydroxy-4-methylpyridine, 1H-3-methylpyrazol-5-one, 1-phenyl-3-methylpyrazol-5-one, and the acid addition salts thereof.

When they are used, the coupler(s) may be present, for example, in an amount ranging from 0.0001% to 10% by weight, such as from 0.005% to 5% by weight, relative to the total weight of the dye composition.

The dye composition in accordance with the present disclosure may also contain various adjuvants conventionally used in hair dye compositions, including, for example, anionic, cationic, nonionic, amphoteric or zwitterionic surfactants or mixtures thereof, anionic, cationic, nonionic, amphoteric or zwitterionic polymers or mixtures thereof, mineral or organic thickeners, antioxidants, penetrants, sequestrants, fragrances, buffers, dispersants, conditioning agents, for instance silicones, film-forming agents, preserving agents and opacifiers.

Of course, a person skilled in the art will take care to select this or these optional additional compound(s) such that the advantageous properties intrinsically associated with the compositions in accordance with the present disclosure are not, or are not substantially, adversely affected by the envisaged addition(s).

The dye composition according to the present disclosure may be in various forms, including, for example, in the form of liquids, creams or gels, or in any other form that is suitable for dyeing keratin fibers, such as human hair.

The nature of the oxidizing agent used in the lightening direct dyeing operation (direct dyeing with an oxidizing agent) or in the oxidation dyeing operation is not critical.

The oxidizing agent may, for example, be chosen from hydrogen peroxide, urea peroxide, alkali metal bromates or ferricyanides, and persalts such as perborates and persulphates. At least one redox enzymes such as laccases, peroxidases and two-electron oxidoreductases (such as uricase) may also be used as oxidizing agent, where appropriate in the presence of the respective donor or cofactor thereof.

According to a non-limiting embodiment of the present disclosure, the process of the present disclosure may be used on hair that has been sensitized by hair treatments other than those of the present disclosure mentioned previously.

Another non-limiting aspect of the present disclosure is a multi-component dyeing agent or kit comprising at least a first component comprising a direct dye composition (A), a second component comprising a composition (B) containing, in a cosmetically acceptable medium, at least one agent for protection against the effects of atmospheric agents, as defined above, and a third component comprising a composition (C) containing, in a cosmetically acceptable medium, at least one zinc-based compound as defined above.

A further aspect of the present disclosure is a multi-component dyeing agent or kit comprising at least a first component comprising a composition (A₁) comprising at least one direct dye, a second component comprising a composition (A₂) containing at least one oxidizing agent, a third component comprising a composition (B) containing, in a cosmetically acceptable medium, at least one agent for protection against the effects of atmospheric agents, and a fourth component comprising a composition (C) containing, in a cosmetically acceptable medium, at least one zinc-based compound as defined above.

Another subject of the present disclosure is a multi-component dyeing agent or kit comprising at least a first component comprising a composition (A₃) containing at least one oxidation base and, optionally, at least one coupler and/or a direct dye, a second component comprising a composition (A₄) containing at least one oxidizing agent, a third component comprising a composition (B) containing, in a cosmetically acceptable medium, at least one agent for protection against the effects of atmospheric agents, and a fourth component comprising a composition (C) containing, in a cosmetically acceptable medium, at least one zinc-based compound as defined above.

The present disclosure will now be illustrated more fully with the aid of the examples that follow, which should not be considered as limiting.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the present disclosure are approximations, unless otherwise indicated the numerical values set forth in the specific examples are reported as precisely as possible. Any numerical value, however, inherently contain certain errors necessarily resulting from the standard deviation found in their respective testing measurements.

By way of non-limiting illustration, concrete examples of certain embodiments of the present disclosure are given below. The amounts of the ingredients are expressed as percentages by weight, relative to the total weight of the composition.

### EXAMPLE 1

### 1) Dyeing Step

At the time of use, a commercial oxidation dye composition, Majirouge 6.66, was mixed with aqueous hydrogen peroxide solution (Professional l'Oréal 20-volumes 6% aqueous hydrogen peroxide solution) weight-for-weight.

The mixture was then applied to locks of permanent-waved hair containing 90% white hairs, at a rate of 10 g of dye mixture/g of lock. The leave-on time was 30 minutes. The locks were then rinsed with water.

The locks were then separated so as to undergo one of the following treatments:

| | | 2-step process | |
|---|---|---|---|
| | | (treatment of | 3-step |
| | 1-step process | oxidation | process |
| | (control treatment | dyeing followed | (treatment of |
| | of conventional | by a photoprotective | the present |
| | oxidation dyeing) | treatment) | disclosure) |
| Setting treatment | 0 | X | X |
| Photoprotective | 0 | 0 | X |
| treatment | | | |

### 2) Setting Treatment Step

The aqueous composition containing one of the zinc salts specified in Table 1 below (composition containing 1% AM with respect to zinc) was applied to certain dyed locks, over their entire length, at a rate of 2 grams per gram of hair.

TABLE 1

| Setting treatment | | Amount of zinc |
|---|---|---|
| applied | Zinc salt applied | salt |
| Treatment C1 | Zinc chloride sold under the reference | 2.0 |
| | 21127-3 by Sigma Aldrich | |
| Treatment C2 | Zinc sulphate sold under the name | 4.2 |
| | 108881 by Merck | |
| Treatment C3 | Zinc gluconate under the name | 6.6 |
| | Givobio G Zn by Seppic | |

The treated locks were then left to stand under a hood for 10 min at 45° C.

### 3) Photoprotective Treatment Steps

The composition 2 specified in Table 2 below was then applied over the entire length of the locks, at a rate of 2 grams per gram of hair.

TABLE 2

| Composition 2 | Amount in grams |
|---|---|
| Benzyl alcohol | 4.0 g |
| Benzoic acid | 0.2 g |
| Benzophenone-4 | 5.0 g |
| Citric acid/trisodium | qs pH = 4 |
| citrate/triethanolamine buffer | |
| Hydroxyethylcellulose | 1.2 g |
| Xanthan gum | 0.4 g |
| Preserving agents | Qs |
| Water | qs 100 |

Next, the treated locks were left to stand under a hood for 10 min at 45° C.

### 4) Removal Step

All the locks were then given a final wash with Dop Camomille® shampoo and then dried under a hood for 10 min at 60° C. Some of the locks that underwent the above treatments were tested for fastness after shampooing and exposure to light. The remaining locks were used as a control (initial dye level without fastness test).

### 5) Steps of Color-Fastness After Washing with Shampoo and Exposure to UV/Visible Rays

A part of the locks that underwent the above treatments was tested for fastness after washing 6 successive times with shampoo and exposure to UV/visible rays.

For the shampoo fastness step, the locks underwent 6 successive washes with Dop Camomille® shampoo with intermediate drying.

For the UV/visible exposure fastness step, the locks were exposed to UV/visible rays for a period of 18 h using a Xenon-lamp sunlight simulator which produced a light spectrum that was reproducible and close to that of sunlight (Suntest XLS sold by the company Atlas).

### 6) Evaluation of the Color Protection

The degradation of the color after this test for fastness following washing and exposure to UV/visible rays, of the treated locks, was evaluated spectrocolorimetrically and compared with dyed locks that have not undergone this test.

These evaluations were accompanied by spectrocolorimetric monitoring. Measurements were taken using a Minolta CM2022 spectrocolorimeter:

The degradation caused by the washing fastness test was expressed as ΔE

ΔE(after test−before test)=√(ΔL*+Δa*²+Δb*²)

The protection was then expressed as a difference in ΔE between the treated and untreated locks. (Positive difference=gain in color protection, negative difference=loss of protection).

### Results:

After exposure to the test for fastness with washing and exposure to UV/visible rays, a considerable degradation of the coloring of the dyed locks having undergone the treatment process A was observed (loss as ΔE=9.9).

It was surprisingly observed that, after this same test, the locks that underwent the 3-step treatment process C (treatments C1, or C2, or C3) exhibited a significant improvement in color protection compared to the locks having undergone only treatment process B (2-step process).

These results were confirmed by colorimetric measurements, which indicated a significant gain in ΔE of the locks C, as compared with the locks B.

Results of Color Degradation after Test for Fastness Following Washing and UV/Visible

| | ΔE compared to the | |
|---|---|---|
| | locks having not | Gain in difference |
| | undergone a | in ΔE compared |
| Lock | fastness test | with treatment B |
| Treatment B | 7.33 | — |
| Treatment C1 | 4.59 | 2.74 |
| Treatment C2 | 4.64 | 2.69 |
| Treatment C3 | 3.14 | 4.19 |

### EXAMPLE 2

### Dyeing Step

At the time of use, the composition of Table 1 below was mixed with aqueous hydrogen peroxide solution (Professional L'Oréal 20-volumes 6% aqueous hydrogen peroxide solution) weight-for-weight.

The mixture was then applied to locks of permanent-waved hair containing 90% white hairs, at a rate of 10 g of dye mixture/g of lock. The leave-on time was 15 minutes on each side of the lock. The coloring was then stopped by rinsing with water.

TABLE 1

| | % | |
|---|---|---|
| Propylene glycol | 8 | g |
| Butoxydiglycol | 9 | g |
| Oleyl alcohol | 6 | g |
| Oleic acid | 3 | g |
| Polyglyceryl-2 oleyl ether | 6 | g |
| Polyglyceryl-4 oleyl ether | 7.7 | g |
| Sodium diethylaminopropyl cocoaspartamide at 55% | 5.5 | g |
| in water (Chimexane HB from Chimex) | | |
| PEG-2 oleamine | 7 | g |
| Alkyl (C13/C15 70/30 50% linear) ether carboxylic acid | 10 | g |
| monoethanolamide (2 EO) | | |
| Pentasodium pentetate at 40% in water | 2.4 | g |
| Ammonium acetate | 0.8 | g |
| 96 degree denatured ethyl alcohol | 7 | g |
| Erythorbic acid | 0.31 | g |
| Phenyl methyl pyrazolone | 0.15 | g |
| Sodium metabisulphite | 0.455 | g |
| p-Phenylenediamine | 6.10⁻⁴ | mol |
| 2,4-Diaminophenoxyethanol HCl | 6.10⁻⁴ | mol |
| 20.5% aqueous ammonia | 10 | g |
| Demineralized water | qs 100 | g |

The locks were then separated so as to undergo one of the following treatments:

| | | B | C | D (present |
|---|---|---|---|---|
| | | 1-step | 1-step | disclosure) |
| | A | process | process | 2-step |
| | no post-dyeing | zinc | filter | process |
| | treatment | treatment | treatment | (Zn + filter) |
| Setting | 0 | X | 0 | X |
| treatment | | | | |
| Photoprotective | 0 | 0 | X | X |
| treatment | | | | |

### Setting Treatment Steps:

The aqueous composition containing zinc gluconate (Givobio G Zn sold by Seppic) was then applied over the entire length of certain dyed locks, at a rate of 2 grams per gram of hair.

| | | D |
|---|---|---|
| | | 1st step of the 2-step process |
| | B | (treatment of the present |
| Composition 1 | 1-step process | disclosure) |
| Zinc gluconate | 30.7 mmol | 14.5 mmol |
| Preserving agents | qs | qs |
| pH agent qs | pH 5 | pH 5 |
| Water | qs 100 g | qs 100 g |

The treated locks were then left to stand under a hood for 10 min at 45° C.

### Photoprotective Treatment Steps:

A photoprotective formula, the composition of which is specified in Table 2 below, was then applied over the entire length of the locks, at a rate of 2 grams per gram of hair.

TABLE 2
Amount in grams

| | Treatment C | Treatment D |
|---|---|---|
| | 1-step process | 2nd step of |
| | Photoprotective | the 2-step |
| Composition 2 | treatment | process |
| Benzyl alcohol | 4.0 g | 4.0 g |
| Benzoic acid | 0.2 g | 0.2 g |
| Benzophenone-4 | 30.7 mmol | 16.2 mmol |
| Citric acid/trisodium | qs pH = 4 | qs pH = 4 |
| citrate/triethanolamine buffer | | |
| Hydroxyethylcellulose | 1.2 g | 1.2 g |
| Xanthan gum | 0.4 g | 0.4 g |
| Preserving agents | qs | qs |
| Water | qs 100 | qs 100 |

The treated locks were then left to stand under a hood for 10 min at 45° C.

### End Step:

All the locks were then given a final wash with Dop Camomille® shampoo and then dried under a hood for 10 min at 60° C. Some of the locks that underwent the above treatments was then tested for fastness after shampooing and exposure to light. The remaining locks served as a control (initial dye level without fastness test).

Steps of Color-Fastness After Washing with Shampoo and Exposure to UV/Visible Rays:

A part of the locks that underwent the above treatments were tested for fastness after washing 6 successive times with shampoo and then exposure to UV/visible rays.

For the shampoo fastness step, the locks underwent 6 successive washes with Dop Camomille® shampoo with intermediate drying.

For the UV/visible exposure fastness step, the locks were exposed to UV/visible rays for a period of 18 hours using a Xenon-lamp sunlight simulator which produced a light spectrum that was reproducible and close to that of sunlight (Suntest XLS sold by the company Atlas).

### Evaluation of the Color Protection:

The degradation of the color after this test for fastness following washing and exposure to UV/visible rays, of the treated locks, was evaluated spectrocolorimetrically and compared with dyed locks that have not undergone this test.

These evaluations were accompanied by spectrocolorimetric monitoring. Measurements were taken using a Minolta CM2022 spectrocolorimeter:

The degradation caused by the washing fastness test was expressed as ΔE

ΔE(after test−before test)=√(ΔL*+Δa*²+Δb*²)

The protection was then expressed as a difference in ΔE between the treated and untreated locks. (Positive difference=gain in color protection, negative difference=loss of protection, significant difference with gain in ΔE>2).

### Results

After exposure to the test for fastness and following washing and exposure to UV/visible rays, a considerable degradation of the coloring of the dyed locks having undergone treatment process A was observed (ΔE=17.7).

Results of the Color Degradation after Test for Fastness Following Washing and UV Exposure (6 Rounds of Shampooing with Dop Camomille®):

| | ΔE compared with | |
|---|---|---|
| | the locks having not | Gain in difference |
| | undergone a | in ΔE compared |
| Lock | fastness test | with the control |
| Treatment A (control) | 17.7 | — |
| Treatment B | 15.4 | 2.3 |
| Treatment C | 16.9 | 0.8 |
| Treatment D (present | 11.7 | 6.0 |
| disclosure) | | |

The calorimetric measurements indicated a significant gain in ΔE of the locks having undergone treatment D as compared with the locks having undergone the other treatments.

It was surprisingly observed that treatment D according to the present disclosure (2-step process) gave a significant improvement in color protection compared with the 1-step treatments, i.e. treatment B or treatment C.

What is claimed is:

1. A process for protecting the color of artificially dyed keratin fibers with respect to washing and atmospheric agents, comprising applying to said artificially dyed keratin fibers:
a) at least one composition (B) comprising, in a cosmetically acceptable medium, at least one agent for protection against atmospheric agents, and
b) at least one composition (C) comprising, in a cosmetically acceptable medium, at least one zinc-based compound;
wherein said at least one composition (C) is applied either before or after dyeing of the said artificially dyed keratin fibers; and said at least one composition (B) is applied after dyeing of said artificially dyed keratin fibers.

2. The process of claim 1, wherein said at least one composition (C) is applied after dyeing of siad keratin fibers, and before or after the application of said composition (B).

3. The process of claim 1, wherein said cosmetically acceptable medium for said at least one composition (B) and said at least one composition (C) may be, independently for each, chosen from water and a mixture of water and at least one cosmetically acceptable organic solvent.

4. The process of claims 1, wherein at least one agent for protection of the keratin fibers from atmospheric agents is chosen from organic UV screening agents, free-radical scavengers and antioxidants.

5. The process of claim 1, wherein said at least one agent for protection of the keratin fibers is chosen from water-soluble or liposoluble organic UV screening agents.

6. The process of claim 5, wherein said at least one water-soluble or liposoluble organic UV screening agent are chosen from dibenzoylmethane derivatives; anthranilates; cinnamic derivatives; salicylic derivatives; camphor derivatives; benzophenone derivatives; triazine derivatives; β,β-diphenylacrylate derivatives; benzotriazole derivatives; benzalmalonate derivatives; benzimidazole derivatives; imidazolines; bis-benzoazolyl derivatives; p-aminobenzoic acid (PABA) derivatives; benzoxazole derivatives; dimers derived from α-alkylstyrene; 4,4-diarylbutadienes and mixtures thereof.

7. The process of claim 6, wherein said at least one agent for the protection of the keratin fibers is at least one liposoluble organic UV screening agent chosen from:
ethylhexyl methoxycinnamate,
butyl methoxydibenzoylmethane,
ethylhexyl salicylate,
benzophenone-3,
n-hexyl 2-(4-diethylamino-2-hydroxy-benzoyl)benzoate,
4-methylbenzylidenecamphor,
1,1-dicarboxy (2,2′-dimethylpropyl)-4,4-diphenyl-butadiene.

8. The process of claim 1, wherein said at least one agent for protection of the keratin fibers from atmospheric agents has a logP of less 6.

9. The process of claim 6, wherein said at least one agent for the protection of the keratin fibers is at least one water-soluble organic UV screening agent are chosen from:
PABA,
PEG-25 PABA
Benzylidenecamphorsulphonic acid,
Camphorbenzalkonium methosulphate,
Terephthalylidenedicamphorsulphonic acid,
Phenylbenzimidazolesulphonic acid,
Disodium phenylbenzimidazoletetrasulphonate,
Benzophenone-4,
Benzophenone-5.
Benzophenone-9, or mixtures thereof.

10. The process of claim 1, wherein said at least one agent for the protection of keratin fibers fom atmospheric agents is present in an amount ranging from 15% to 50% by weight, relative to the total weight of the composition.

11. The process of claim 1, wherein said at least one zinc-based compound is chosen from water-soluble zinc salts.

12. The process claim 11, in which said at least one water-soluble zinc salt is chosen from zinc sulphate, zinc chloride, zinc lactate, zinc gluconate, zinc phenolsulphonate, zinc salicylate and its derivatives, or mixtures thereof.

13. The process of claim 1, wherein said at least one zinc-based compound is present in an amount ranging from 0.005% to 30% by weight, relative to the total weight of the composition (C).

14. The process of claim 1, wherein said composition (B) and/or said composition (C) further comprise at least one aromatic alcohol and at least one aromatic carboxylic acid, or a salt thereof.

15. The process of claim 1, wherein said composition (B) and/or said compostion (C) further comprises at least one conditioning agent.

16. The process of claim 15, wherein said at least one conditioning agent is present in an amount ranging from 0.001% to 20% by weight, relative to the total weight of the composition of which it is part.

17. The process of claim 1, wherein at least one of said composition (B) and said composition (C) is in the form of an aqueous lotion, an aqueous-alcoholic lotion, an oil, a gel, a milk, a cream, an emulsion or a mousse.

18. The process of claim 1,further comprising, before or after application of at least one of said composition (B) and said composition (C), at least one step of rinsing and/or a step of washing with shampoo.

19. The process of claim 1, further comprising, after application of at least one of said composition (B) and said composition (C), at least one additional step of total or partial drying of the artificially dyed keratin fibers.

20. The proscess of claim 1, further comprising at least one step of heating at least one said composition (B) and said composition (C) to form at least one heated composition, and applying said at least one heated composition to said artificially dyed keratin fibers.

21. The process of claim 1, further comprising a step of heating at least one of said composition (B) and said composition (C), after or during the application of said composition(s) to said artificially dyed keratin fibers.

22. A process for dyeing keratin fibers, comprising applying to said fibers:
(i) at least one direct and/or oxidation dye composition (A) in the presence of at least one oxidizing agent for a period of time sufficient to develop a desired color,
(ii) at least one composition (B) comprising, in a cosmetically acceptable medium, at least one agent for protection against atmospheric agents,
(iii) at least one composition (C) comprising, in a cosmetically acceptable medium, at least one zinc-based compound;
wherein:
said at least one composition (C) is applied before or after the application of said at least one direct and/or oxidation dye (A); and
said at least one composition (B) is applied after the application of said at least one direct and/or oxidation dye composition (A).

23. The process of claim 22, wherein said at least one composition (C) is applied to said keratin fibers after application of said at least one direct and/or oxidation dye composition and at least one oxidation agent, and before or after apllication of said at least one composition (B).

24. The process of claim 22, wherein the application of said at least one composition (A) is followed by rinsing and/or drying of the keratin fibers.

25. The process of claim 22, wherein application of at least one of said at least one composition (B) and said at least one composition (C) is followed by rinsing and/or drying of the keratin fibers, and/or by heating of the keratin fibers.

26. The process of claim 22, wherein at least one of said at least one composition (B) and said at least one composition (C) is preheated.

27. The process of claim 22, wherein at least one of said at least one composition (B) and at least one composition (C) is applied after the application of the direct or oxidation dye composition (A), either immediately or after a delay, wherein the applications of said compositions are optionally repeated between two colorings.

28. A multi-component dyeing agent or kit comprising:
at least a first component comprising a direct dye composition (A);
at least one second component comprising at least one composition (B), said at least one composition (B) comprising, in a cosmetically acceptable medium, at least one agent for the protection of keratin fibers against atmospheric agents; and
at least one third component comprising at least one composition (C) comprising, in a cosmetically acceptable medium, at least one zinc-based compound.

29. A multi-component dyeing agent or kit comprising:
at least one first component comprising at least one composition (A₁), said at least one composition (A₁) comprising at least one direct dye;
at least one second component comprising a composition (A₂), said at least one composition (A₁) comprising at least one oxidizing agent;
at least one third component comprising at least one composition (B), said at least one composition (B) comprising, in a cosmetically acceptable medium, at least one agent for the protection of keratin fibers against atmospheric agents; and
at least one fourth component comprising at least one composition (C), said at least one composition (C) comprising, in a cosmetically acceptable medium, at least on zinc based compound.

30. A multi-component dyeing agent or kit comprising:
at least a first component comprising at least one composition (A₃), said at least one composition (A₃) comprising at least one oxidation base and, optionally, at least one coupler and/or at least one direct dye;
at least one second component comprising at least one composition (A₄), said at least one composition (A₄) comprising at least one oxidizing agent;
at least one third component comprising at least one composition (B), said at least one composition (B) comprising, in a cosmetically acceptable medium, at least one agebt for the protection of keratin fibers against atmospheric agents; and
at least one fourth component comprising at least one compostion (C), said at least one composition C comprising, in a cosmetically acceptable medium, at least one zinc-based compound.
